(19) United States
(12) Patent Application Publication
(10) Pub. No.: US 2026/0272706 A1
(43) Pub. Date: Sep. 17, 2026

(54) FULL BODY THERMAL THERAPY ASSEMBLY AND SYSTEM

(71) Applicants: Jaime Sanchez Solana, Culver City, CA (US); Zonghu Zhu, Xiamen City (CN)

(72) Inventors: Jaime Sanchez Solana, Culver City, CA (US); Zonghu Zhu, Xiamen City (CN)

(21) Appl. No.: 19/561,038

(22) Filed: Mar. 9, 2026

Related U.S. Application Data
(60) Provisional application No. 63/770,242, filed on Mar. 11, 2025.

(51) Int. Cl. A61F 7/00; A61F 7/02

(52) U.S. Cl. CPC A61F 7/0085; A61F 2007/0018; A61F 2007/0029; A61F 2007/0039; A61F 2007/0056; A61F 2007/0091; A61F 2007/0233

(57) ABSTRACT

A thermal therapy system that includes a temperature control unit with a refrigerant loop, a water tank system, at least one water pump configured to circulate water between the water tank system and a wearable garment; and an air pump. The system also includes a wearable garment configured to be worn by a user that includes one or more garment portions, each garment portion including an air bag, and a water bag disposed inwardly of the air bag. The water bag defines a plurality of water channels configured to receive temperature-conditioned water from the temperature control unit. The air bag is configured to receive pressurized air from the air pump and to inflate to press the water bag into contact with the user's body. The system also includes a plurality of hoses fluidly connecting the temperature control unit to the garment, including at least one water inlet hose, at least one water outlet hose, and at least one air hose.

### CROSS REFERENCE TO RELATED APPLICATIONS

This application claims the benefit of U.S. Provisional Application No. 63/770,242, filed on Mar. 11, 2025, the entirety of which is incorporated by reference herein.

### FIELD OF THE INVENTION

The present invention relates to a full body thermal therapy assembly and system.

### BACKGROUND OF THE INVENTION

The current market only offers traditional cold plunges that are bulky, require maintenance and are pricy in most cases. They also require a certain level of mobility to get in and out and they present a risk specially for people with reduced mobility.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the invention there is provided a thermal therapy system that includes a temperature control unit with a refrigerant loop, a water tank system, at least one water pump configured to circulate water between the water tank system and a wearable garment, and an air pump, a wearable garment configured to be worn by a user, the garment includes a plurality of garment portions, each garment portion including an outer fabric layer, an air bag disposed inwardly of the outer fabric layer, and a water bag disposed inwardly of the air bag. The water bag defines a plurality of water channels configured to receive temperature-conditioned water from the temperature control unit, the air bag is configured to receive pressurized air from the air pump and to inflate to press the water bag into contact with the user's body, and a plurality of hoses fluidly connecting the temperature control unit to the garment, including at least one water inlet hose, at least one water outlet hose, and at least one air hose.

The refrigerant loop includes a compressor, a four-way reversing valve, a heat sink, and a plate heat exchanger, and where the four-way reversing valve is configured to reverse the direction of refrigerant flow to switch the temperature control unit between a cooling mode and a heating mode. The water tank system includes a large water tank and a small water tank fluidly connected through a restricted interconnection having a flow area substantially smaller than a cross-sectional area of either tank. The small water tank includes a one-way divider configured to permit water flow in a first direction and to substantially prevent water flow in an opposite direction. The one-way divider may include a pivoting trapdoor. The temperature control unit further includes an ice-making coil disposed within the water tank system, the ice-making coil being in thermal communication with the refrigerant loop such that ice forms around the ice-making coil when refrigerant is directed therethrough. The temperature control unit is configured to route return water from the garment through a region containing ice formed around the ice-making coil before the return water enters the water tank system. In an embodiment, water returning from the garment goes through the cooling cycle before going back into the tank via the ice making coil. The at least one water pump includes an internal circulation pump configured to circulate water between the water tank system and a plate heat exchanger, and at least one garment pump configured to circulate water through the garment. The at least one garment pump includes a first garment pump configured to circulate water through a left body portion and a left arm sleeve of the garment, and a second garment pump configured to circulate water through a right body portion and a right arm sleeve of the garment. The water channels are configured as water highways extending in a substantially vertical direction when the garment is worn by the user, with water inlets positioned proximate the user's shoulders and water outlets positioned proximate the user's feet. The air bag and the water bag are joined together at intermittent weld points disposed at spaced intervals, such that inflation of the air bag presses the water bag into contact with the user's body without substantially occluding the water channels. The system may include a remote controller connected to the temperature control unit, the remote controller including at least one control button, and a biometric sensor configured to sense at least one biometric parameter of the user.

In accordance with another aspect of the invention there is provided a temperature control unit for a thermal therapy system, the temperature control unit including a refrigerant loop comprising a compressor, a four-way reversing valve, a heat sink, and a plate heat exchanger, where the four-way reversing valve is configured to reverse the direction of refrigerant flow to switch the temperature control unit between a cooling mode and a heating mode; a water tank system comprising a large water tank and a small water tank fluidly connected through a restricted interconnection, where the small water tank includes a one-way divider configured to permit water flow in a first direction and to substantially prevent water flow in an opposite direction; an ice-making coil disposed within the water tank system, the ice-making coil being in thermal communication with the refrigerant loop through a dedicated capillary branch; a plurality of water pumps including an internal circulation pump and at least one garment pump; and an air pump configured to supply pressurized air to a wearable garment.

The one-way divider includes a pivoting trapdoor that opens under flow pressure in the first direction and closes under flow pressure in the opposite direction. The restricted interconnection has a flow area configured to limit migration of water between the large water tank and the small water tank such that heating effects are substantially confined to the small water tank during a heating mode. The ice-making coil includes a helical metal tube disposed within an ice-making tank that is formed as a subdivision of the large water tank. The temperature control unit is configured to form ice around the ice-making coil when water in a region surrounding the ice-making coil is substantially static. The temperature control unit is configured to route return water from a garment through a region containing ice formed around the ice-making coil before the return water enters the large water tank or the small water tank. The temperature control unit may also include a three-way purge valve configured to selectively connect the air pump to at least one water inlet hose to force residual water from a garment back to the water tank system. The temperature control unit may include one or more water valve blocks integrating multiple valves for controlling water flow through the temperature control unit. The temperature control unit may include insulation surrounding the water tank system. The temperature control unit may include a liquid storage tank disposed between the four-way reversing valve and the compressor, the liquid storage tank configured to store excess refrigerant during transitions between heating and cooling modes. The liquid storage tank for the refrigerant could also be between the heat sink and the capillary tubes for the transition between water cooling and the ice making.

In accordance with another aspect of the invention there is provided a thermal therapy system that includes a temperature control unit including a refrigerant loop and a water tank system; an ice-making coil disposed within the water tank system, the ice-making coil being in thermal communication with the refrigerant loop through a dedicated capillary branch, where ice forms around the ice-making coil when refrigerant is directed through the ice-making coil while water surrounding the ice-making coil is substantially static; a wearable garment fluidly connected to the temperature control unit; and a water circulation path configured to route return water from the garment through a region containing ice formed around the ice-making coil, such that heat is extracted from the return water by the ice before the return water enters the water tank system.

The ice-making coil may be disposed within an ice-making tank formed as a subdivision of a larger water tank. The ice-making coil may be disposed within a region of a unified water tank, and where baffles or flow guides direct return water through the region containing the ice. The dedicated capillary branch includes a second capillary tube and at least one valve configured to selectively direct refrigerant through the ice-making coil. The ice formed around the ice-making coil serves as a thermal battery that stabilizes water temperature during circulation through the garment.

In accordance with another aspect of the invention, there is provided a method of operating a thermal therapy system that includes the steps of (a) cooling water in a water tank system of a temperature control unit; (b) after the water is cooled, a user donning a wearable garment fluidly connected to the temperature control unit, the garment comprising a water bag defining a plurality of water channels and an air bag disposed outwardly of the water bag; (c) inflating the air bag to press the water bag into contact with the user's body; (d) after the air bag is at least partially inflated, circulating the cooled water from the water tank system through the water channels of the water bag; (e) after completion of a therapy session, removing water from the water bag; and (f) deflating the air bag.

The water may be cooled by circulating the water through a heat exchanger in the temperature control unit. The method may include, prior to step (b), forming ice around an ice-making coil disposed within the water tank system. The method may include during step (d), return water from the garment is routed through a region containing the ice formed around the ice-making coil such that heat is extracted from the return water by the ice. The ice formed around the ice-making coil may serve as a thermal battery that maintains a stable water temperature throughout step (d). Step (e) may include purging residual water from the water bag by directing pressurized air into the water channels to force the residual water back to the water tank system. Step (e) may include actuating a three-way purge valve to connect an air pump to at least one water inlet hose of the garment. The pressurized air in step (e) may enter the water channels through shoulder inlet connections and force the residual water toward foot outlet connections or vice versa. Step (f) may be performed after step (e) such that the air bag is deflated after water is removed from the water bag. Step (c) may be completed before step (d) such that the water bag is in substantial contact with the user's body before cooled water is introduced into the water channels. Step (a) may be performed during a pre-conditioning period before the user dons the garment in step (b). During step (d), cooled water may enter the garment at shoulder inlet connections proximate the user's shoulders and flows downwardly through the water channels toward foot outlet connections proximate the user's feet or vice versa or at other locations throughout the garment.

In accordance with another aspect of the invention there is provided a temperature control unit for a thermal therapy system, the temperature control unit including a refrigerant loop configurable between a cooling mode and a heating mode; a plate heat exchanger in thermal communication with the refrigerant loop; a large water tank; a small water tank fluidly connected to the large water tank through a restricted interconnection having a flow area that limits migration of water between the large water tank and the small water tank; a one-way divider disposed within the small water tank, the one-way divider configured to permit water flow in a first direction and to substantially prevent water flow in an opposite direction; and at least one water pump configured to circulate water through the plate heat exchanger and selectively through the large water tank or the small water tank; where, in the heating mode, water flow is directed preferentially through the small water tank such that heating effects are substantially confined to the small water tank while the large water tank remains at a lower temperature.

The one-way divider includes a pivoting trapdoor having a hinge axis, sealing surfaces, and an opening bias in the first direction. The one-way divider prevents backflow or back-mixing of heated return water into a supply side of the small water tank. The small water tank includes approximately one-third of a total water capacity of the temperature control unit, and where the large water tank includes approximately two-thirds of the total water capacity. The restricted interconnection is sized to substantially prevent mixing of water between the large water tank and the small water tank during heating or cooling operations. The configuration of the large water tank, the small water tank, the restricted interconnection, and the one-way divider enables rapid transitions between the heating mode and the cooling mode by drawing upon a cold water reservoir maintained in the large water tank after heating water in the small water tank.

In accordance with another aspect of the invention there is provided a wearable garment for a thermal therapy system, the garment including a plurality of garment portions configured to cover substantially the entire body of a user, each garment portion including an outer fabric layer; an air bag disposed inwardly of the outer fabric layer; and a water bag disposed inwardly of the air bag; where the water bag defines a plurality of water channels configured as water highways extending in a substantially vertical direction when the garment is worn by the user; shoulder inlet connections positioned proximate the user's shoulders for receiving temperature-conditioned water; foot outlet connections positioned proximate the user's feet for returning water; and where the air bag and the water bag are joined together at intermittent weld points disposed at spaced intervals, such that inflation of the air bag presses the water bag into contact with the user's body without substantially occluding the water channels.

The plurality of garment portions includes a right body portion, a left body portion, a right arm sleeve, and a left arm sleeve. The right arm sleeve and the left arm sleeve are attached to the right body portion and the left body portion, respectively, at shoulder connections. Each body water bag defines at least four substantially parallel water channels extending from a shoulder inlet region to a foot outlet region. The water channels are defined by weld lines formed by welding inner and outer surfaces of the water bag together along elongated lines. The intermittent weld points are aligned with weld lines of the water bag such that the weld points coincide with walls between adjacent water channels. The air bag includes a plurality of internal weld lines that extend generally horizontally across the air bag when the garment is worn, each weld line terminating at each end in an enlarged circular weld point. The weld lines create a series of substantially parallel chambers within the air bag that prevent excessive ballooning during inflation. The air bag includes a non-inflating portion in a chest region that corresponds to an overlapping flap region of the garment, the non-inflating portion configured to prevent double-layer inflation when the garment is closed around the user. The garment may include at least one emergency air release valve positioned at an accessible location on the garment. The garment may include closures including leg closures extending along an inner leg seam and chest closures extending along a front of the garment, where the closures comprise hook-and-loop fasteners. The garment is configured to open fully for donning, doffing, and cleaning. The water channels bifurcate at the shoulder inlet connections to provide separate flow paths to arm water bags and body water bags.

In accordance with another aspect of the invention there is provided water bag for a wearable garment of a thermal therapy system, the water bag including a flexible, substantially water-impermeable material defining a plurality of water channels configured to receive temperature-conditioned water; weld lines formed by welding inner and outer surfaces of the water bag together along elongated lines, the weld lines defining boundaries of the water channels; and a plurality of internal weld points disposed within the water channels.

The spacing between adjacent internal weld points may vary by body region such that: in regions of high bending, the spacing between adjacent internal weld points is increased to permit greater flexibility and water flow; and in regions of lower bending, the spacing between adjacent internal weld points is decreased to balance pressure distribution across the water bag. The internal weld points may be discrete spot welds that join inner and outer surfaces of the water bag together at spaced locations within the water channels. The regions of high bending may include at least one of knees, hips, and elbows. The regions of lower bending may include a torso region. The spacing between adjacent internal weld points in high-bending regions may be in the range of approximately 20 mm to 40 mm. The spacing between adjacent internal weld points in lower-bending regions may be in the range of approximately 10 mm to 25 mm.

In accordance with another aspect of the invention there is provided thermal therapy system that includes a temperature control unit including a water tank system and an air pump; a wearable garment fluidly connected to the temperature control unit, the garment including at least one water bag defining a plurality of water channels; at least one water inlet hose connecting the temperature control unit to the garment; at least one water outlet hose connecting the garment to the temperature control unit; and a three-way purge valve configured to selectively connect the air pump to the at least one water inlet hose; where, when the three-way purge valve is actuated, pressurized air from the air pump is directed into the at least one water inlet hose to force residual water remaining in the water channels back through the at least one water outlet hose and into the water tank system.

The three-way purge valve may be configured to connect the air pump to an air hose for inflating an air bag in the garment when the three-way purge valve is in a first position, and to connect the air pump to the at least one water inlet hose when the three-way purge valve is in a second position. The purging of residual water from the garment reduces weight of the garment for handling and storage. The purging of residual water from the garment returns water to the water tank system for re-conditioning. The purging of residual water from the garment ensures that a subsequent therapy session begins with freshly conditioned water at garment inlets. The pressurized air enters the water channels through shoulder inlet connections and forces residual water toward foot outlet connections. The air pump is configured to supply pressurized air at a pressure and flow rate sufficient to purge substantially all residual water from the water channels without damaging the garment.

In accordance with another aspect of the invention there is provided method of operating a thermal therapy system for cold therapy, the method including the steps of: donning a wearable garment, the garment comprising a water bag defining a plurality of water channels and an air bag disposed outwardly of the water bag; activating an ice-making mode to form ice around an ice-making coil disposed within a water tank system of a temperature control unit, where water surrounding the ice-making coil is substantially static during ice formation; activating a water cooling mode to pre-cool water in the water tank system by circulating water through a plate heat exchanger in a closed internal loop; activating a garment inflation mode to inflate the air bag and press the water bag into contact with a user's body; activating a cool water flow mode to circulate cold water from the water tank system through the water channels of the garment, where return water from the garment is routed through a region containing ice formed around the ice-making coil such that heat is extracted from the return water by the ice; and upon completion of a therapy session, activating a garment deflation mode and a water return mode to deflate the air bag and purge residual water from the water channels.

The ice-making mode is performed during a pre-conditioning period while the user is donning the garment and before the therapy session begins. The garment inflation mode is performed before the cool water flow mode such that the air bag is inflated and the water bag is pressed into contact with the user's body prior to introduction of cold water. Cold water enters the garment at shoulder inlet connections proximate the user's shoulders and flows downwardly through the water channels toward foot outlet connections proximate the user's feet. Purging residual water includes actuating a three-way purge valve to connect an air pump to at least one water inlet hose and supplying pressurized air to force residual water from the water channels back to the water tank system.

In accordance with another aspect of the invention there is provided method of operating a thermal therapy system for contrast therapy, the method including the steps of: donning a wearable garment fluidly connected to a temperature control unit, the temperature control unit including a large water tank and a small water tank fluidly connected through a restricted interconnection, the small water tank including a one-way divider; performing cold therapy for a first period by circulating cold water from the large water tank through the garment; transitioning to a heating mode by directing water flow preferentially through the small water tank while the large water tank remains at a lower temperature, the one-way divider preventing back-mixing of heated return water into a supply side of the small water tank; performing hot therapy for a second period by circulating heated water from the small water tank through the garment; transitioning back to a cooling mode by drawing upon cold water maintained in the large water tank; and repeating the alternating cold therapy and hot therapy as desired.

The restricted interconnection limits migration of water between the large water tank and the small water tank such that heating effects are substantially confined to the small water tank. The small water tank includes approximately one-third of a total water volume of the temperature control unit. The one-way divider includes a pivoting trapdoor that opens to permit water flow toward a supply side and closes to prevent water flow from a return side toward the supply side. Transitioning from hot therapy to cold therapy is accomplished rapidly due to the cold water reservoir maintained in the large water tank during hot therapy.

In accordance with another aspect of the invention there is provided method of operating a thermal therapy system with closed-loop biometric control, the method including the steps of: donning a wearable garment fluidly connected to a temperature control unit; positioning a biometric sensor on a user, the biometric sensor being integrated into a remote controller connected to the temperature control unit; activating a therapy mode to circulate temperature-conditioned water through the garment; sensing at least one biometric parameter of the user using the biometric sensor; and automatically adjusting at least one operating parameter of the thermal therapy system responsive to the sensed biometric parameter.

The at least one biometric parameter may include at least one of heart rate, heart rate variability, respiratory rate, blood oxygen saturation, and skin temperature. The at least one biometric parameter may be used to infer a core body temperature of the user. The at least one operating parameter may include water temperature. The at least one operating parameter may include session duration. Automatically adjusting includes reducing a water temperature or terminating the therapy session when the user's heart rate exceeds a threshold value or extending a session duration when heart rate variability indicates an inadequate physiological response. The remote controller is connected to the temperature control unit through a controller wire routed through a hose connecting the temperature control unit to the garment, the controller wire emerging at a location proximate the user's wrist or hand.

The present invention relates generally to thermal therapy systems for the human body, and more particularly to an integrated thermal therapy system comprising a temperature control or cooling and heating unit fluidly coupled to a wearable garment configured to circulate temperature-conditioned water over substantially the entire body of a user while an inflatable air layer presses the water layer into uniform contact with the user's skin. It will be appreciated that cooling and heating unit is often used herein, however, this is not a limitation on the unit or the invention and the unit may only providing heating, may only providing cooling or may provide both. Accordingly, temperature control unit is also used herein.

In one aspect, the present invention provides a thermal therapy system comprising a cooling and heating unit or temperature control unit and a wearable garment. The cooling and heating unit may include a refrigerant loop having a compressor, a four-way reversing valve, a heat sink, and a plate heat exchanger. The cooling and heating unit further includes a water tank system comprising a large water tank and a small water tank fluidly connected to the large water tank through a restricted interconnection. The small water tank includes a one-way divider configured to permit water flow in a first direction and to substantially prevent water flow in an opposite direction. The cooling and heating unit may also include an ice-making tank having an ice-making coil disposed therein. The ice making coil may be disposed within the large water tank. The ice-making coil is in thermal communication with the refrigerant loop through a dedicated capillary branch such that, when refrigerant is directed through the ice-making coil while water in the ice-making tank is substantially static, ice forms around the ice-making coil. When water is subsequently circulated through the ice-making tank, heat is extracted from the circulating water by the ice. The cooling and heating unit further includes a plurality of water pumps, including an internal circulation pump for circulating water between the water tank system and the plate heat exchanger, and at least one garment pump for circulating water through the garment. An air pump is provided for inflating one or more air bags in the garment and for selectively purging residual water from the garment back to the water tank system through a three-way valve arrangement.

The wearable garment may include a plurality of garment portions configured to cover substantially the entire body of a user. Each garment portion includes an outer fabric layer, an air bag disposed inwardly of the outer fabric layer, and a water bag disposed inwardly of the air bag. The water bag defines a plurality of water channels configured as water highways extending in a substantially or generally vertical direction (with respect to the wearer) when the garment is worn by a user, with water inlets positioned proximate the user's shoulders and water outlets positioned proximate the user's feet. In another embodiment, the water highways may extend in any direction, not necessarily vertically, such as horizontally, diagonally or the like. The air bag and the water bag are joined together at intermittent weld points such that inflation of the air bag presses the water bag into contact with the user's skin without substantially occluding the water channels. The water and air bags may be joined by any method, including welding (e.g., ultrasonic welding), adhesive, sewing, snaps, velcro or the like.

In another aspect, the present invention provides a cooling and heating unit for a thermal therapy system comprising a refrigerant loop, a water tank system including a large water tank and a small water tank with a one-way divider, an ice-making tank with an ice-making coil, a plurality of water pumps, and an air pump. The ice-making coil may or may not be contained in a separate tank. It will be appreciated that the coil is preferably in direct contact with the water that is in the big tank and that water may be forced from the garment to be in contact with the ice cube to maximize heat absorption. The refrigerant loop is configurable between a cooling mode and a heating mode through a four-way reversing valve. In the cooling mode, water is cooled in the plate heat exchanger and ice is formed in the ice-making tank. In the heating mode, water is heated in the plate heat exchanger, with flow directed preferentially through the small water tank such that only a portion of the total water volume is heated, thereby enabling rapid transitions between heating and cooling modes.

In yet another aspect, the present invention provides a wearable garment for a thermal therapy system comprising a plurality of garment portions, each garment portion including an outer fabric layer, an air bag, and a water bag with water channels configured as substantially vertical water highways. The garment includes water inlet connections proximate the shoulders and water outlet connections proximate the feet, such that temperature-conditioned water flows from the shoulders toward the feet when circulating through the garment. The garment is configured to open fully for donning and doffing, with closures including straps and tabs that may include Velcro or other temporary closure mechanism positioned along the legs and chest. It will be appreciated that in another embodiment, the garment not cover the full body. For example, the garment may be a three-quarter garment that only covers up to the chest and no arms. In another embodiment, the garment may only cover or be directed to only the arms or only the legs.

In still another aspect, the present invention provides methods of operating a thermal therapy system in various modes, including an ice-making mode, a water cooling mode, a garment inflation mode, a cool water flow mode, a garment deflation mode, a water return mode, a water heating mode, and a hot water flow mode.

The present invention provides several advantages over prior art thermal therapy systems. The integrated ice-making subsystem enables the system to deliver an immediate cold sensation upon initial water circulation and to maintain a stable low water temperature throughout a therapy session by utilizing the latent heat of the ice. The two-tank architecture with the one-way divider and restricted interconnection enables rapid transitions between heating and cooling modes by confining the heated water to the small tank while preserving a cold reservoir in the large tank. The air-pump purge feature enables residual water in the garment to be returned to the cooling and heating unit at the end of a session, reducing garment weight and ensuring that the next session begins with freshly conditioned water. The water highway configuration with shoulder inlets and foot outlets provides rapid transit of temperature-conditioned water and delivers the coldest water to the physiologically significant torso region. The intermittent coupling between the air bag and water bag prevents occlusion of the water channels during inflation while ensuring uniform body contact. A remote controller with integrated biometric sensing enables closed-loop control of the thermal therapy system responsive to the user's physiological state.

The present invention is a thermal suit or garment that includes a water circulating system configured to deliver various thermal experiences to the body to achieve a wide variety of benefits both physical and psychological. In one preferred embodiment, the thermal regulation system includes a garment configured to be worn by a user and a cooling unit fluidly coupled to the garment via hoses. The garment includes a water circulation network designed to distribute temperature-regulated water across substantial portions of the user's body, thereby simulating various thermal experiences such as traditional cold plunges and saunas without requiring the user to get wet or utilize bulky equipment. The cooling unit is responsible for regulating the temperature of the water, capable of both cooling and heating, and circulating it through the garment, enabling a compact, portable form factor that allows usage in diverse settings, such as on a couch, bed, or chair, to integrate thermal therapy seamlessly into daily routines.

The system is engineered to deliver a wide array of physical and psychological benefits, including but not limited to muscle recovery, mental recovery, stress relief, improved sleep, enhanced metabolic and nervous system health, and potential applications in treating conditions such as diabetes, improving testosterone levels, alleviating depression, and lowering blood pressure. These benefits are achieved by replicating the “cold shock” response in cold therapy sessions and providing customizable heat therapy, with the garment ensuring even distribution of thermal effects across the body.

In preferred embodiments, the system incorporates a biofeedback system, integrated into a remote control device. This system monitors key biometric parameters in real-time, such as heart rate (HR), heart rate variability (HRV), respiratory rate, blood oxygen saturation, core body temperature, and others, via sensors embedded in the remote that the user holds during sessions. Based on these readings, algorithms dynamically adjust session parameters, including water temperature, duration, and frequency, to personalize treatments. For instance, the system detects the initial sympathetic nervous system activation (e.g., HR increase due to cold shock, activating brown fat, vasoconstriction, and shivering), followed by a transition to parasympathetic activation (e.g., HR decline as the body enters energy-saving mode). The water temperature is modulated accordingly, maintaining lower temperatures during the peak HR phase to maximize therapeutic effects and gradually adjusting as HR stabilizes, to optimize outcomes while enhancing safety, particularly for users with varying physiological responses, such as differences between males and females in optimal temperature, time, and frequency.

The garment is constructed with a double-layer design comprising separate air and water chambers. The air chamber inflates to provide pneumatic compression, improving contact between the water layer and the user's skin for efficient thermal transfer. The water chamber circulates the temperature-regulated fluid. To optimize this construction, the air and water layers are connected via a third intermediary layer with intermittent welding points, which prevent wrinkling of the water layer during air inflation that could otherwise restrict water flow and compromise uniform coverage. This intermittent welding allows the air chamber to expand without unevenly compressing the water chamber, resulting in improved body contact and thermal efficacy, especially in full-body garments where such issues are amplified compared to smaller competitors' devices. In a preferred embodiment, the two layers are connected to one another and there is a third layer, which is the outer fabric layer. The intermittent welding areas are preferably part of the air layer. In a preferred embodiment, the air chambers are inflated first to ensure proper contact with the body and then the water flows through the garment. It will be appreciated that if it was not done in this order, the cold shock would not occur because the water only starts flowing once the air chambers are inflated. It is important that the air chambers do not block the water ways, which why the intermittent welding points are relevant.

In a preferred garment configuration, the overall structure maintains a generally constant cross-sectional area along the vertical (Y) axis to ensure uniform pressure distribution. The water flow is primarily vertical, divided into multiple channels, preferably eight (however, this is not a limitation), to minimize horizontal flow, which can slow circulation, increase temperature gradients, and reduce the cold sensation. Various water entry and exit designs have been developed, including foot-to-foot, shoulder-to-foot, and foot-to-shoulder paths. The preferred embodiment utilizes water entry from the shoulders or upper body area and exit from the feet, capitalizing on human physiology: cooling vital organs in the upper body first induces vasoconstriction in extremities, reducing the total heat load to be removed and enabling replication of cold plunge effects with a smaller, quieter cooling unit that preferably uses significantly less water. An inner tube within the garment shortens the water return path, aiding efficient heat capture and return to the cooling unit while facilitating residual water drainage post-session. The water path is not limiting. In another embodiment, the water may enter near the feet and exit near the shoulders or upper body. Further, the inner tube within the garment preferably shortens the water inlet as well as the return path, not just the return path.

The distance between welding points in the water chamber varies by body area: increased in high-bending regions (e.g., joints) to allow greater water volume and flow, and decreased in high-flow areas to balance pressures and prevent blockages or reflux. Connectors are strategically positioned in challenging flow areas, such as the feet and trapezius region (connecting the main body to arm sleeves), to ensure adequate water distribution, prevent pinching or bending, and provide secondary escape paths in case of obstructions. The foot connector, for example, serves as a backup drainage point in bending-prone areas.

For adjustability and ease of use, the garment employs Velcro closures on legs, arms, and chest, allowing fit for different body sizes, overlap of layers to eliminate gaps in coverage, and full opening for easy donning and doffing, critical for commercial applications like clinics or gyms. In one embodiment, chest closures feature straps extending from one torso side through loopholes on the opposite side, secured with additional neck Velcro, improving torso contact over previous designs. However, this is not limiting and only exemplary. The Velcro straps may not go through loop holes and simply connect with the other side. This contrasts with zipper alternatives, which may create gaps and hinder cleanability. The garment is preferably constructed as a main body piece with detachable arm sleeves, minimizing separate components (e.g., three total parts) to maximize coverage and simplify assembly.

The cooling unit features a multi-channel system for efficient temperature regulation, addressing challenges in cooling the body despite its heat generation and enabling rapid transitions between cold and hot therapies. In preferred operation, the unit supports three modes via a circulation valve and valves controlling flow paths: 1. Cooling water mode: A closed loop between the heat exchanger and an insulated tank pre-cools the full water volume (e.g., 12 liters) to a target low temperature, such as 2° C. (35° F.), prior to session start, ensuring immediate cold shock upon circulation, unlike competitors that cool during flow, leading to delays and reduced efficacy. This pre-cooling replicates the skin temperature drop of a 10° C. (50° F.) immersion plunge. 2. Heating water mode: The circulation valve releases a reduced volume (e.g., 4 liters) into a closed loop with the heat exchanger, excluding the main tank, for faster heating with lower energy use. The volume can also be released to a separate tank. 3. Water flowing mode: Once at target temperature, garment valves open for circulation through the garment, with parallel internal channels (tank-heat exchanger) maintaining cooling or heating.

For transitions (e.g., cold to hot), the tank valve closes to heat only the circulating volume, leveraging body heat to accelerate the process. Post-hot session, mixing heated water with remaining cold tank water enables quick return to cooling. The circulation valve also supports future scalability, such as connecting multiple garments or adapting to smaller accessories (e.g., knee wraps) by regulating water volume, reducing pre-conditioning times.

To manage residual water post-session, a common issue in circulating systems, a three-way valve connects an air pump to the water channels, transforming them temporarily into air pathways to force water back into the tank. This minimizes uncooled residual water, enhances cold shock in subsequent sessions, and lightens the garment for easier handling.

User-independent operation is facilitated by a handheld remote control (that may be placed on a finger like a ring or on a wrist) with an intuitive interface, allowing control without reaching the cooling unit. Session setup occurs via the unit's display or app (e.g., selecting treatment type, time, temperature), followed by garment donning. The remote then enables: (1) air inflation for skin contact; (2) water flow initiation; and (3) emergency stops, which halt circulation and deflate air chambers. The remote also houses the biometric sensors.

Safety features include dual air release valves positioned above the hips for easy access, enabling rapid manual decompression. Additional elements in preferred embodiments encompass: a removable, foldable water tank for enhanced portability in future models; integrated power lines in hoses to support add-on technologies (e.g., sensors, LEDs, vibration motors) without additional connections; auditory and visual cues (sounds, lights) for immersive relaxation guidance; cleaning aids like custom sponges and UV sterilization structures; commercial variants with one unit supporting multiple garments; L-shaped hose connectors to minimize bending and kinking; modular garment configurations (e.g., without sleeves or legs); and zoned pneumatic compression via air pumps and valves to create pressure gradients simulating a standing position (higher lower-body pressure), potentially enhancing results over lying-down use, while also providing general compression benefits.

The cooling unit layout optimizes performance: water pumps are positioned at the tank's lowest level for maximum efficiency and external to the tank to avoid heat transfer to the water. In a preferred embodiment, three pumps are employed, one for internal cycling (tank-heat exchanger) and two for garment sides (right and left), with water inlets at each foot to split flow bilaterally and further reduce horizontal circulation.

Water circulation paths may include exiting the cooling unit and traveling through a tube or pipe under or adjacent the water chambers (and air chamber) to near the top of the garment where the water enters the water chamber near the shoulder and then exits near or at the foot.

The present invention may include a cooling device that includes a garment assembly that includes an inner fluid member that defines a fluid chamber configured to receive cooling fluid, and an outer air member that defines an outer air chamber configured to receive air, where an inner layer of the inner fluid member defines a cooling cavity configured to receive a body or a user, and an outer cover that defines a cover interior, where the garment assembly is received in the cover interior. When the outer air chamber is inflated and the inner fluid chamber is filled with cooling fluid, the outer air chamber pushes the inner fluid member against the user positioned in the cooling cavity.

The present invention may include a cooling device that includes an upper fluid member that defines an upper fluid chamber configured to receive cooling fluid, a lower fluid member that defines a lower fluid chamber configured to receive cooling fluid, where the upper fluid member and lower fluid member cooperate to define a cooling cavity therebetween, whereby the cooling cavity is configured to receive a body or a user, and a lower air member that defines an air chamber configured to receive air, whereby when the lower air chamber is inflated and the lower fluid chamber is filled with cooling fluid, the lower air member pushes the lower fluid member against the user positioned in the cooling cavity.

The present invention is a cooling device or dry cold plunge. Generally, the device includes a cooling unit connected to a cover or garment that may be shaped like a sleeping bag and that is configured to move cooling fluid (e.g., water) throughout the garment and around the user's body to replicate the feeling of water immersion or cold plunge. The cooling unit may also be used with smaller garments that are associated with specific body parts, such as the leg, arm, and back.

In a preferred embodiment, the garment or cooling assembly includes a double layer structure that includes inner and outer layers or members. The inner member (closest to the body) is filled with fluid or water (and may be referred to as the fluid or water member) and the outer member is filled with air (and may be referred to as the air member), which helps replicate the feeling of full body water immersion. During use, in order for the user to feel the cold, the water chamber or inner member is preferably in direct contact with the user's skin. By filling the outer member with air, the inner or fluid member is pushed toward and against the user, which can move to difficult to reach areas, such as the inner thigh or armpits. The air chamber construction pushes the water towards the body simulating the effect of water immersion.

The air chamber or air member acts as cushion. Given the size of the cooling assembly and the cooling unit the cooling assembly may be used on the floor and may include at least a portion of the air chamber under the body, which may help keep the water chamber in contact with the body and create comfort for the user. The airbag or air portion may also act as a size adjustment system. The cooling unit preferably includes one or more pumps that inflate the air bags up to a certain or predetermined pressure and the ability to cool the water and pump the water into the fluid or water chamber(s) of the cooling device. One or more hoses to direct the air and water from the cooling unit to the garment or assembly may be included. Depending on the size of the user, the airbag will inflate more or less ensuring optimum or desired “fit” every time. The airbag pushes and moves the water bag so that it molds to the user's body. In other words, it creates negative space around the user's body or at least a portion thereof. In an embodiment, multiple hoses and/or units may be used to fill different portions of the air and water chambers or to fill different air or water chambers that are associated with different portions of the body (e.g., separate air and/or water chambers for one or more arms, one or more legs, the torso, the midsection, etc.).

One feature of an embodiment of the present invention is optimizing the amount of time it takes for the air chambers to inflate. This is critical in achieving the desired “cold shock.” If the chambers are inflated too slowly, the body may not feel the cold water all at once and by the time there is full contact the body may already be accustomed to the cold. Three different solutions to achieve an inflation time of around 60 seconds or less are presented. One is to use multiple pumps or multiple cooling units. Reduce the amount of air needed by adding Velcro or other closure straps around the body. Adding user adjustable and/or operable straps to the legs and/or arms may reduce the inflation time. For example, the inclusion of two-three straps may achieve the desired result. Lastly, the air inflation may be programed or predetermined based on the size of the user. For example, the user may enter their weight and height and the device will preset to the specific size by inflating the chambers before the user gets in assembly. Furthermore, the cooling unit may include more than one tank (e.g., first and second tanks) so that the tanks can be cooled or heated separately. This can allow a general holding tank and then a tank for a single instance of using and filling the cooling device. The hoses can also include electrical wires and/or connections to provide power for the remote or other components in the cooling device.

An aspect of the present invention or dry cold plunge is the ability to precisely control water temperature of the fluid or the temperature transmitted to the user at all times. Traditional cold plunges lack this control due to their large water volume and open-air construction, which leads to significant heat loss, especially given the large surface area exposed to external or room temperature. One of the fundamental aspects of cold therapy is allowing the body to gradually restore its natural temperature without external assistance. In traditional cold plunges, the temperature shift is binary. Meaning the user if either in or out of the water. In the present invention, once a session ends, the system may gradually raise the water temperature, encouraging the body to warm up independently.

In a preferred embodiment, the system may include sensors (e.g., temperature sensors) in both the water tank (in the pump unit or a separate water unit) and in the cooling assembly or garment itself. This allows precise temperature regulation and creates a closed-loop system for a personalized experience tailored to individual needs. Cold therapy serves various purposes, from pain management and muscle recovery to potential benefits in metabolic health, including reducing insulin resistance, as some studies suggest. As a result, different use cases require different or specific protocols, adjusting time, temperature, and temperature gradients. The present invention may provide customization to meet these varying needs. One relevant application may be sleep induction, since the body naturally cools down before sleep. The system may include a cycle that gently lowers body temperature by two to three degrees, the optimal range for promoting sleep onset. The system can use hot or warm fluid or water to provide other effects (such as recovery) as opposed to cold fluid.

An example of a cycle of using the present invention is presented. This cycle or protocol is only exemplary and may be adjusted and predefined by the user via an app or the display of the device based on needs or desires of the result. Step 0 or the pre-step may include cooling the fluid in the tank from room temperature to approximately 5° C. or 42° F. This may be done via a control on the cooling assembly, on the cooling unit/pump or on an app. This may take 5-10 minutes. Step 1 includes the user getting in the cooling assembly or garment and exposing the full body to the cold temperature. This step may be 3-10 minutes. The system may include, for example, three modes which include different temperatures so the user can adjust based on their liking or tolerance. For example, mode one may be 15° C. or 59° F., mode two may be 12° C. or 54° F., and mode three may be 8-10° C. or 46 to 50° F. Step 2 may be 5-10 minutes and may include where the water stops cooling down but the user stays in the cooling device to keep the exposure to a cold environment, thereby forcing the body to warm up naturally. In this scenario, the temperature increases slowly (due to ambient conditions) and forces the body to reheat. During step 2, the water rises to about 20° C. or 68° F., at which point the cycle ends and the user exits the cooling assembly.

Steps to those above or separately may be added to aid in delivering rapid cold sensation to the user, which replicates the sensation of cold water immersion. The further steps introduce a different dynamic for inflating the air and water chambers. This may include inflating the air chambers first and then start pumping the water. The steps include cooling down the water in the tank (e.g., 5-10 minutes or 5-30 minutes or even longer). Cooling down the water may include the user turning on the device. The water preferably stays in the tank, thus accelerating the cooling pace and leaving the garment at room temperature. Next, the air chambers are inflated (e.g., 2-3 minutes). The user enters the garment and the air chambers inflate to fill the gaps between the body and the chambers. The water chambers remain empty. Next, one or more of the various modes described above may be used. With the user inside and the air chambers fully inflated, the water pump is activated and the cold water flows through the water chambers or water path of the garment (e.g., feet to chest to arm and the returning to the feet), thus replicating the feeling of stepping into cold water (e.g., 3-10 minutes). Next, the water temperature rises from the cold temperature of the previous step to room or ambient temperature (e.g., 5-10 minutes). In this step the water cooling stops and the user stays in the garment forcing the body to warm up naturally. The water can also be warmed gradually. The temperature can be set to match the room temperature to provide a pleasant feeling when exiting the garment. In the next step, when the cycle ends, the air is released and the water is pushed back into the tank. This would generally happen before the user exists the garment because it will be a lot easier. The user could exit as this step is taking place as well, no need to wait. The user then exits the garment.

It will be appreciated that a benefit of the present invention includes safety benefits, such as it is safer entering the cooling assembly, compared to entering liquid cold plunges, due to the barrier a user needs to overcome to enter and also the slippery nature of being around a large volume of water. The present invention may lay on the ground, thus minimizing the risk of falling and making it a much better option for older demographics as well as physically challenged individuals.

In another embodiment of the invention, the present invention is a cooling assembly that may include a top water or fluid member or bag, that is covered by a top fabric or outer layer, a bottom water or fluid member or bag, a bottom air bag or member positioned below the bottom fluid member and a bottom fabric or layer. The top and bottom fluid members cooperate to define a cooling cavity therebetween that is configured to receive or fit a user. In short, this embodiment includes a top set of layers and a bottom set of layers. The bottom layer set of layers includes the bottom air member and the bottom fluid member. In use, the bottom layers cover or are associated with the bottom of the user (e.g., bottom of legs, buttocks, back and arms). The bottom layers are also associated with some or all of the vertical surfaces of the user's body (e.g., the sides of legs and arms, the side of the core, and the sides of the inner thigh and shoulders or armpit). The top or upper fluid member acts as a blanket filled with water covering the horizontal surfaces of the top of the body. An upper air member and chamber can also be included. Furthermore, a single air member and a single fluid member that together surround the user's body (or define the cooling cavity) may be included.

Within the water chamber, there is a water path and various portions or channels within the upper and lower water chambers or the water chamber with the weld walls or weld points there-between. The water inlet and outlet may be located anywhere on the water or fluid member(s). These various channels and paths help prevent “bagging” and help direct the water as desired. The paths and channels shown are not limiting.

It will be appreciated that each of the dots shown in the drawings is a weld point or spot and the undulating wave-type sections (or weld wall) are weld areas that provide a wall or the like that directs the water as desired (i.e., these are the borders of the water or fluid channels. The distance between weld points or dots can be increased or decreased to provide faster or slower water flow and more or less coverage on the user's body.

There may be another embodiment of the cooling assembly that includes an inner structure or garment and an outer cover. Similar to the other embodiments described herein, the inner garment includes an outer or air member and an inner or fluid member and a cooling cavity therebetween where the user is positioned during use. The cooling assembly may include first and second halves or hemispheres. In a preferred embodiment, the cooling assembly includes a water channel that preferably does not get blocked during use and uses water efficiently thus allowing an efficient pump. The water chamber may include multiple welding points to help prevent water collection and “bagging” within the chamber. In one embodiment of the invention, the cooling assembly may be divided into the two halves, hemispheres, sections or portions (e.g., right and left sections). Each half or section may include one or more channels that extend from the foot area all the way to the arm and back and down to the foot again or vice versa. In a preferred embodiment, each half or side section of the garment may include a separate water stream or path and these separate paths may be powered by two or more separate pumps. It will be appreciated that the right side may include the same water path and each side may have its own cooling unit/pump. A water path in each half reduces the travelling distance of the water and the area of coverage before returning to the cooling unit. These shorter cycles allow cooling down the water more frequently giving better temperature control and higher overall efficiency.

In an embodiment, the arm sleeve may be an independent piece so the user can move and close the device by themself. The exemplary steps include: enter through the top and insert legs all the way in, insert arms through the arm sleeves, close the Velcro on the vest or chest area, lay back before closing the zipper, close the zipper and press the start button to start the cycle. However, this is only exemplary.

If the user is utilizing the assembly alone, the airbag may not be inflated until the user is in the cooling cavity. In a preferred embodiment, a remote may be positioned inside where the remote is accessible by the user. The remote may preferably also shut down the device if needed at any given point. The remote may include a start/stop button and may be connected to the air hose. If the user has an emergency they may pull from the remote (to unplug) and the device may automatically shut down and the air is released so the user can get out.

The cooling assembly 10 may include an inner wearable garment or garment assembly, which may be a vest portion and pants portion that are inserted into the outer cover, which is similar to a sleeping bag. The assembly may be embodied in a full body suit with two leg portions, a vest portion and arm sleeves connected to the main vest portion at or near the shoulders. When using the inner garment, the user may insert their legs first, then their arms into the arm sleeves before closing the zipper. The legs may be connected to the main body or vest portion. The arms may be loose or separate so the user can move more freely to close the Velcro or other closure.

In a preferred embodiment, the garments wrap around the body in a single layer rather than having separate upper and a lower layers or members. This may simplify the construction but also may allow for better coverage.

### BRIEF DESCRIPTION OF THE DRAWINGS

FIG. 1 is a bottom perspective view of the garment in a closed position together with the cooling unit and air and water hoses;

FIG. 2 is a top perspective view of the garment in an open position together with the cooling unit and air and water hoses;

FIG. 3 is an exploded view of the garment showing the water bag above, the air bag, above the outer fabric layer for both the right arm, right body portion, left arm and left body portion, together with the various water inlet, water outlet and air hoses;

FIG. 4A is an exploded plan view of the left arm air bag, left body air bag, right arm air bag and right body air bag;

FIG. 4B is an exploded plan view of the left arm water bag, left body water bag, right arm water bag and right body water bag;

FIG. 5 is a schematic cross-sectional view of a water bag, air bag and outer fabric layer prior to being welded together;

FIG. 6 is a schematic cross-sectional view of a water bag, air bag and outer fabric layer after being welded together at one end, and including weld points;

FIG. 7 is a plan view of the left and right water bags (including left and right body water bags and left and right arm water bags) connected to the cooling unit via hoses and showing the water path from the cooling unit, through the inlet hoses, through the water highways or channels, through the outlet hoses and back to the cooling unit;

FIG. 8A is a side cross-sectional view of a user's leg in a leg section of the garment prior to inflation;

FIG. 8B is a side cross-sectional view of a user's leg in a leg section of the garment after inflation;

FIG. 9 is a partial exploded view of the cooling unit;

FIG. 10 is a flow diagram of the system;

FIG. 10A shows the refrigerant cycle during the ice making process;

FIG. 10B shows the water cooling process, including the water cycle and refrigerant cycle;

FIG. 10C shows the garment inflation process for the air bags, including the air cycle;

FIG. 10D shows the cool water flow process including the water cycle or path and the refrigerant cycle;

FIG. 10E shows the garment deflation process for the air bags, including the air cycle;

FIG. 10F shows water return process for any excess water left in the water bags, including the air cycle;

FIG. 10G shows the water heating process, including the water cycle and refrigerant cycle;

FIG. 10H shows the hot water flow process including the water cycle or path and the refrigerant cycle;

FIG. 11 is a perspective view of the garment with some of the straps and tabs in the open position

FIG. 12 is a perspective view of the garment with all straps and tabs in the closed position; and

FIG. 13 is a perspective view of the remote.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward” and “below” used herein are merely for ease of description and refer to the orientation of the components as shown in the figures. It should be understood that any orientation of the components described herein is within the scope of the present invention.

### Overview of the Thermal Therapy System.

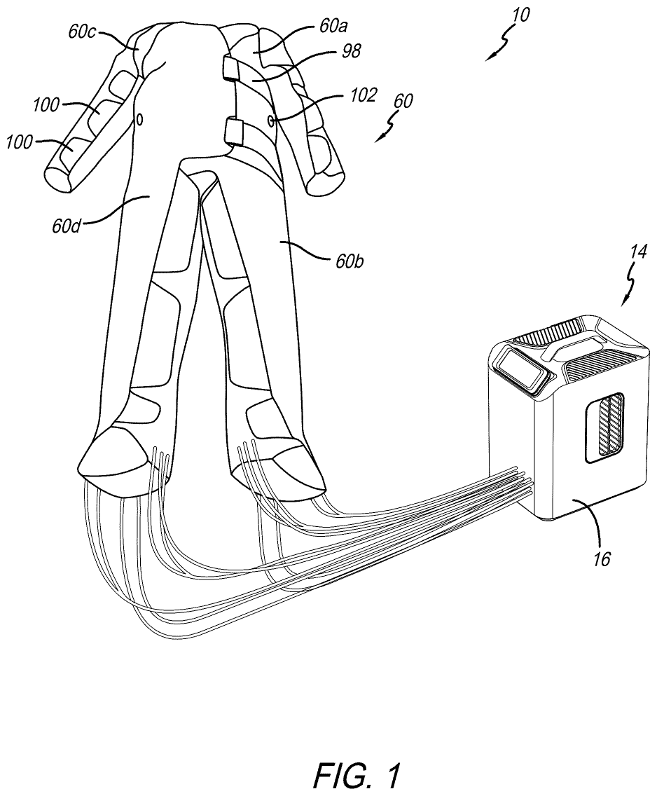

Referring now to FIGS. 1-3, there is shown a thermal therapy system 10 in accordance with a preferred embodiment of the present invention. The thermal therapy system 10 comprises a cooling and heating unit 14 and a wearable garment 60. The cooling and heating unit 14 is fluidly coupled to the garment 60 through a plurality of hoses, including water inlet hoses 80, 82, water outlet hoses 84, 86, and an air hose 88. The cooling and heating unit 14 is configured to condition water to a desired temperature and to circulate the temperature-conditioned water through the garment 60. The cooling and heating unit 14 is also configured to supply pressurized air to the garment 60 to inflate one or more air bags disposed within the garment 60.

As shown in FIG. 1, the garment 60 is depicted in a closed position suitable for wearing by a user. The garment 60 substantially covers the user's body from the shoulders to the feet, including both arms. The cooling and heating unit 14 is positioned adjacent to the garment 60, and the hoses 80, 82, 84, 86, 88 extend from the cooling and heating unit 14 to various connection points on the garment 60.

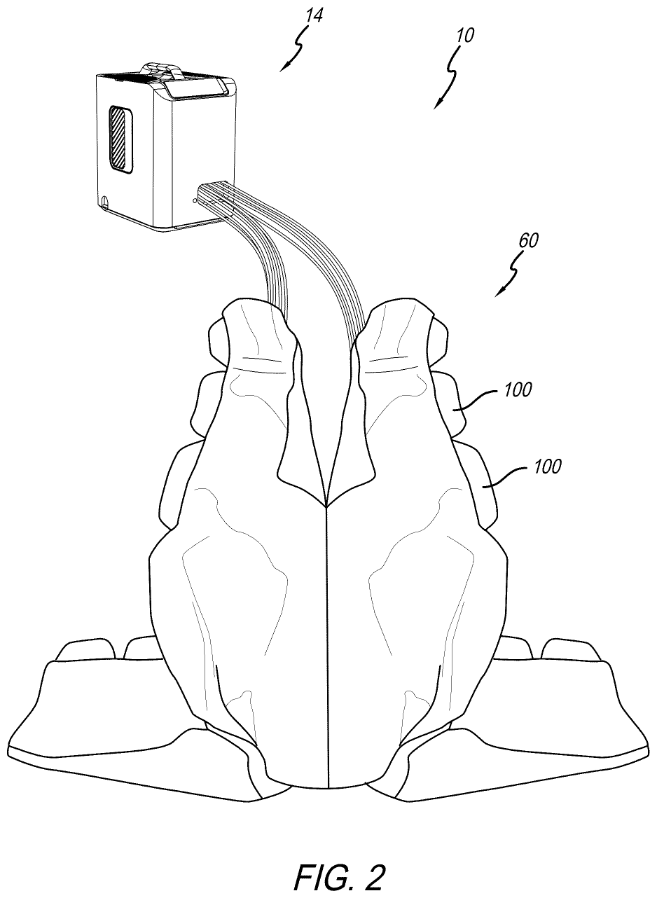

As shown in FIG. 2, the garment 60 is depicted in an open position suitable for donning by a user. The garment 60 is configured to open fully such that a user may lie down upon the opened garment 60 and subsequently close the garment 60 around the user's body. This fully openable configuration facilitates ease of donning and doffing and enables cleaning of the interior surfaces of the garment 60.

### Garment Construction and Layer Assembly.

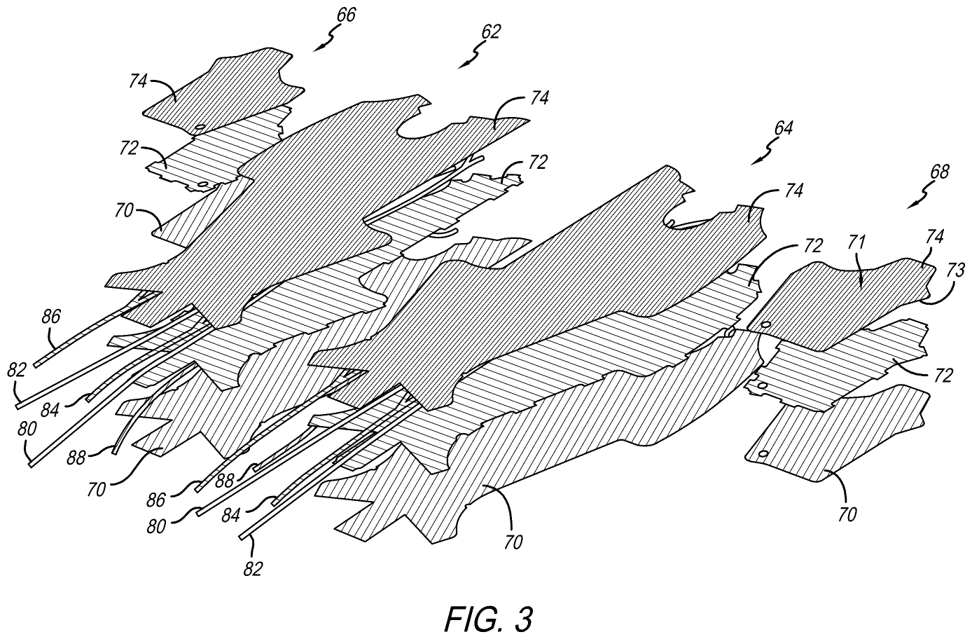

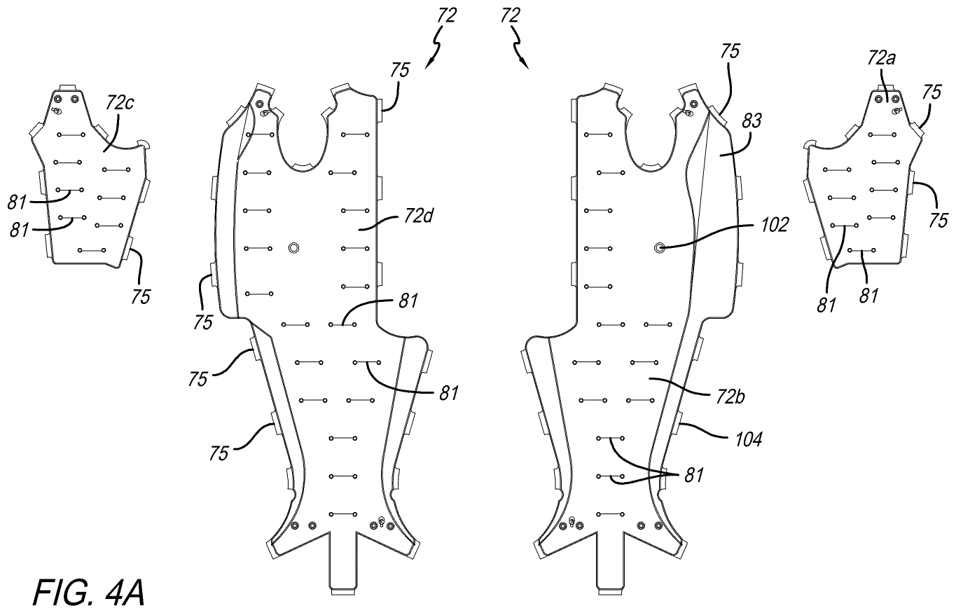

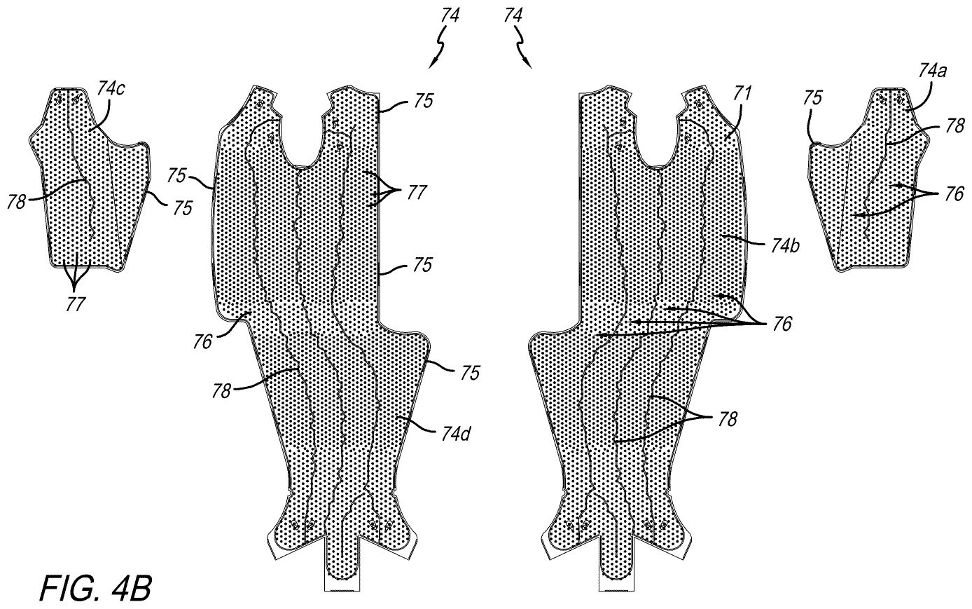

Referring now to FIGS. 3-4B, there is shown an exploded view of the garment 60 illustrating the layered construction thereof. The garment 60 comprises a plurality of garment portions configured to cover corresponding portions of a user's body. In the illustrated embodiment, the garment 60 comprises a right body portion 62, a left body portion 64, a right arm sleeve 66, and a left arm sleeve 68. It will be appreciated that the garment 60 may be configured with additional or fewer garment portions as desired for particular applications.

Each garment portion comprises three primary layers: an outer fabric layer 70, an air bag 72 disposed inwardly of the outer fabric layer 70, and a water bag 74 disposed inwardly of the air bag 72. When the garment 60 is worn by a user, the water bag 74 is positioned closest to the user's skin, the air bag 72 is disposed between the water bag 74 and the outer fabric layer 70, and the outer fabric layer 70 forms the outermost layer of the garment 60. It will be appreciated that the cross-hatching on the layers in FIG. 3 is included to differentiate the various layers and not to indicate a cross-section.

The outer fabric layer 70 is preferably formed from a durable, flexible fabric material that is comfortable against the user's skin on the exterior and that provides structural support for the air bag 72 and water bag 74. The outer fabric layer 70 may comprise a woven or non-woven fabric, and may include moisture-wicking or antimicrobial properties as desired.

The air bag 72 is preferably formed from a flexible, substantially air-impermeable material such as a thermoplastic polyurethane (TPU) film, polyethylene, or other suitable polymer material. The air bag 72 preferably includes an inner layer 67 and outer layer 69. The air bag 72 is configured to receive pressurized air from the air pump 44 of the cooling and heating unit 14 and to inflate to a desired pressure. When inflated, the air bag 72 expands inwardly toward the user's body, thereby pressing the water bag 74 into contact with the user's skin.

The water bag 74 is preferably formed from a flexible, substantially water-impermeable material such as a thermoplastic polyurethane (TPU) film, polyethylene, or other suitable polymer material. The water bag 74 preferably includes an inner layer 71 and outer layer 73. The water bag 74 defines a plurality of water channels 76 (see FIG. 4B) through which temperature-conditioned water flows when circulated by the cooling and heating unit 14. The water channels 76 are preferably formed by welding the inner surfaces of the inner layer 71 and outer layer 73 of the water bag 74 together along elongated weld walls or weld lines 78, thereby defining discrete channels between the weld lines.

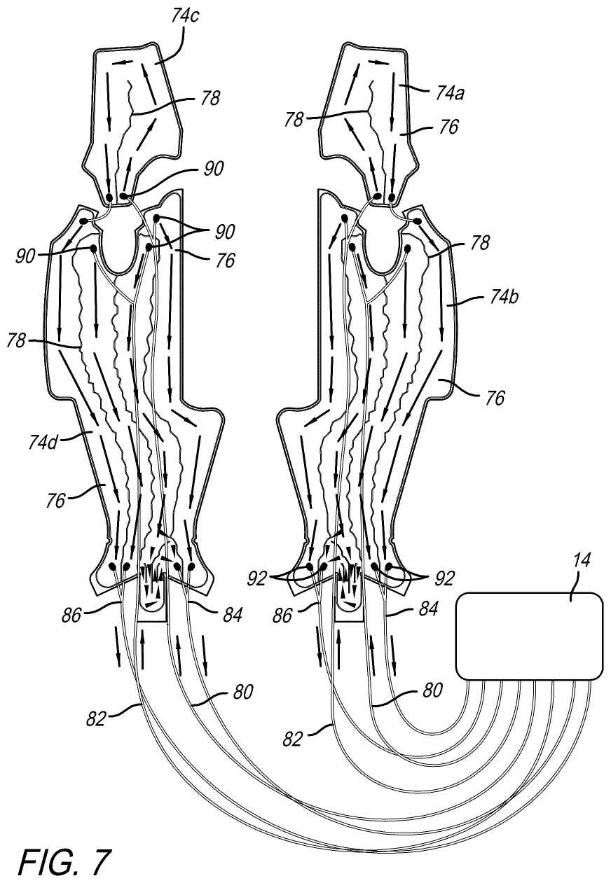

As shown in FIG. 3, the garment 60 includes water inlet hoses 80, 82 for supplying temperature-conditioned water from the cooling and heating unit 14 to the garment 60, water outlet hoses 84, 86 for returning water from the garment 60 to the cooling and heating unit 14, and one or more air hose(s) 88 for supplying pressurized air from the cooling and heating unit 14 to the air bags 72 in the garment 60. As shown in FIG. 7, in a preferred embodiment, the water inlet hoses 80, 82 (which may split or divide as necessary) are connected to the garment 60 at shoulder inlet connections 90 positioned proximate the user's shoulders when the garment 60 is worn. The water outlet hoses 84, 86 (which may split or divide as necessary) are connected to the garment 60 at foot outlet connections 92 positioned proximate the user's feet when the garment 60 is worn. This arrangement ensures that the coldest water enters the garment 60 at or near the shoulders, proximate the user's vital organs and core, and flows downwardly through the water channels 76 toward the feet, providing maximum thermal effect at the physiologically significant torso region. In another embodiment, the water cycle could be reversed and the water may enter via the feet and exit via shoulders. In other embodiments, one or more water inlet hoses may be positioned or may enter the garment (connect to the water channels therein) at a first location on the garment 60 and one or more water outlet hoses may be positioned or may exit the garment (connect to the water channels therein) at a second location on the garment that is spaced from the first location. Essentially, the water inlet and water outlet may be at two different locations. It will be appreciated that the inlet or outlet may be positioned adjacent the shoulder, which height (with respect to the feet, where the opposite outlet or inlet may be positioned) or positioning allows coverage of the entire body except for the neck and head. The shoulder is an exemplary height or location. The upper body positioning may also be at or near the chest or upper back. Furthermore, as shown in FIG. 7, the water inlet hose 80 may bifurcate or divide to provide a water inlet at two different locations. FIG. 7 shows the exemplary water paths utilizing the four water channels in each of the body water bags. At least a portion of one of the water paths extends through the arm water bag. It will be appreciated that the water path for body water bag 74b and arm water bag 74a is essentially the same as the water path for body water bag 74d and arm water bag 74c shows water. The arrows in FIG. 7 represent the water paths starting from the temperate control unit, through the inlet hoses 80, 82, through the body and arm water bags, through the outlet hoses 84, 86 and back to the temperature control unit 14. FIG. 7 shows inlet hose 80 bifurcating and connecting to one water channel 76 near the shoulder on the body water bag 74d and connecting to the arm water bag 74c. The water path extends around arm water bag 74c and then a hose routes the water from the arm water bag 74c to one of the outer water channels 76 in the body water bag 74d. Water inlet hose 82 connects to two different locations (two water channels 76) near the shoulders on the body water bag 74d. The water from both inlet hoses 80 and 82 then flows down the four water channels 76 and toward the feet where the water exits through the outlet hoses 84 and 86 (which each bifurcate to receive water from two channels). It will be appreciated that the portions of the garment that contain or comprise the right body water bag 74d, right arm water bag 74c, left body water bag 74b and left arm water bag 74a may be referred to as the right body portion 60d, right arm portion 60c, left body portion 60b and left arm portion 60a, respectively (see FIG. 1). The air path may also include hose interconnections between the arm and body air bags.

### Air Bag and Water Bag Configuration

Referring now to FIGS. 4A and 4B, there are shown exploded plan views of the air bags 72 and water bags 74, respectively, for the various garment portions. FIG. 4A illustrates the left arm air bag 72a, left body air bag 72b, right arm air bag 72c, and right body air bag 72d. FIG. 4B illustrates the left arm water bag 74a, left body water bag 74b, right arm water bag 74c, and right body water bag 74d.

Each air bag 72 is configured to substantially correspond in shape and size to the garment portion in which it is disposed. The air bags 72 may be configured as separate, discrete components for each garment portion, or may be fluidly interconnected such that pressurized air supplied through the air hose 88 inflates all air bags 72 simultaneously. In one embodiment, the air bags 72 are fluidly interconnected through internal passages or external manifolds to ensure substantially uniform inflation pressure across all garment portions. The air bags 72 preferably also include weld tabs 75 that correspond with weld tabs 75 on the water bags 74.

Referring again to FIG. 4A, each air bag 72 includes a plurality of internal weld lines 81 that extend generally horizontally across the air bag 72 when the garment 60 is worn by a user. The weld lines 81 are formed by welding the two layers 67 and 69 of the air bag 72 together along elongated lines, thereby creating a series of substantially parallel chambers or cells within the air bag 72. Each weld line 81 preferably terminates at each end in an enlarged circular weld point, which provides a robust termination that resists tearing or stress concentration at the ends of the weld line. The weld lines 81 serve to prevent excessive ballooning of the air bag 72 when inflated, ensuring that the air bag 72 expands in a controlled, substantially uniform manner rather than forming a single large balloon. By constraining the expansion of the air bag 72 into a series of cells, the weld lines 81 help maintain a relatively flat profile of the inflated air bag 72, which in turn ensures more uniform pressure distribution against the water bag 74 and, ultimately, against the user's body. It will be appreciated that the weld lines 81 in the air bag 72 are horizontal to help maximize water coverage. If they were vertical (parallel to the water channel welding lines) it may force the water to flow through the airbag welding lines and not the rest of the garment. However, this is not a limitation and in another embodiment, the weld lines 81 may be diagonal, vertical or other arrangement.

The configuration of the weld lines 81 is substantially symmetric between the left and right body portions of the garment 60, with one notable exception in the chest area. Referring to FIG. 4A, the left body air bag 72b includes a non-inflating portion 83 in the chest region that does not contain weld lines 81 and does not receive air during inflation. The non-inflating portion 83 is defined by the boundary of the air bag 72d, such that the air bag material in this region remains substantially flat and does not inflate. The non-inflating portion 83 corresponds to the overlapping flap region of the garment 60 that closes over the opposing chest portion when the garment 60 is secured around a user. Because the right body air bag 72d in this region will already be inflated and in contact with the user's chest when the garment 60 is closed, having a second inflated air layer on top of the first would create excessive pressure on the user's chest. The non-inflating portion 83 therefore prevents this double-layer inflation effect, ensuring user comfort while still providing full coverage when the garment 60 is closed. Additionally, the absence of weld lines 81 around the valve 102 helps air escape more rapidly from the air bag 72 during deflation, thereby reducing the time required to fully deflate the garment 60.

Each water bag 74 defines a plurality of water channels 76 configured as water highways extending in a substantially vertical direction when the garment 60 is worn by a user. As used herein, the term “water highways” refers to elongated water channels that extend substantially along the length of the garment portion with minimal horizontal deviation, thereby providing a rapid, direct flow path for temperature-conditioned water. The water highways are configured to minimize turns, bends, and horizontal traversals, thereby maintaining pressure uniformity across the garment 60 and ensuring rapid transit of water to deliver stable temperature-conditioned water to all areas of the user's body.

In the illustrated embodiment, each body water bag 74b, 74d defines four substantially parallel water channels 76 or highways extending from near a shoulder inlet region to near a foot outlet region. At the shoulder inlet region, the water channels 76 bifurcate to provide a separate flow path to the corresponding arm water bag 74a, 74c while continuing the primary flow path down the body water bag 74b, 74d. The arm water bags 74a, 74c are connected to the body water bags 74b, 74d at shoulder connections such that water entering the shoulder inlet flows both into the arm sleeve and down the body portion. FIG. 7 includes arrows that show the actual water paths through the various water channels 76, connecting hoses, etc.

The weld lines 78 defining the water channels 76 are configured with spacing that varies by body region to balance flexibility and flow characteristics. The distance between welding lines 78 helps maintain the width throughout the water channels 76 to keep the pressure relatively constant. The weld points 77, and the distance between them, help keep the pressure balanced.

Referring again to FIG. 4B, in addition to the weld lines 78 that define the boundaries of the water channels 76, each water bag 74 includes a plurality of internal weld points 77 disposed within the water channels 76. The internal weld points 77 are discrete spot welds that join the inner and outer layers 71 and 73 of the water bag 74 together at spaced locations within the water channels 76. The internal weld points 77 are distinct from the weld lines 78, which form elongated, substantially continuous welds that define the channel boundaries and direct water flow along the water highways.

The internal weld points 77 serve several functions. First, the internal weld points 77 prevent excessive expansion or “bagging” of the water bag 74 when filled with water under pressure. Without the internal weld points 77, the water channels 76 could expand into balloon-like shapes when pressurized, which would reduce the percentage of water in contact with the skin. By constraining the expansion of the water bag 74 at discrete locations, the internal weld points 77 maintain a relatively flat profile of the water bag 74 against the user's body, thereby maximizing the contact area for thermal transfer.

Second, the internal weld points 77 influence the flow characteristics of water within the water channels 76. The spacing between adjacent internal weld points 77 affects the flow velocity and distribution of water within each channel. A greater distance between internal weld points 77 permits a larger cross-sectional flow area within the channel, resulting in reduced flow resistance and increased flow rate. Conversely, a smaller distance between internal weld points 77 reduces the cross-sectional flow area, resulting in increased flow resistance and reduced flow rate. By varying the spacing of the internal weld points 77, the flow characteristics within different regions of the water bag 74 can be tuned to achieve desired performance objectives.

Third, the internal weld points 77 help maintain pressure balance across the water bag 74. Without appropriate flow balancing, water could preferentially flow through certain channels or regions while bypassing others, resulting in uneven thermal coverage. The internal weld points 77, by influencing flow resistance in different regions, help ensure that water is distributed substantially uniformly across all areas of the water bag 74.

In a preferred embodiment, the spacing of the internal weld points 77 may vary by body region to accommodate the different functional requirements of each region (this is evident in FIG. 4B). In regions of high bending, such as the knees, hips, elbows, and other joints, the distance between adjacent internal weld points 77 is increased. The increased spacing in these high-bending regions provides two advantages. First, the greater distance between internal weld points 77 permits a larger cross-sectional flow area, allowing more water to pass through the channel even when the channel is partially compressed during bending. This ensures that water flow is maintained during user movement. Second, the increased spacing provides greater flexibility in the water bag 74, enabling the garment 60 to conform more readily to the user's body during bending movements without restricting water flow or causing discomfort.

In regions of lower bending but higher flow demand, such as the torso, the distance between adjacent internal weld points 77 may be reduced. The reduced spacing in these regions increases the flow resistance, which helps balance the overall pressure distribution across the water bag 74. Without this balancing, water could preferentially flow through shorter or less restrictive paths, resulting in inadequate coverage of certain body areas. By reducing the spacing between internal weld points 77 in high-flow regions, the flow resistance in these regions is increased to more closely match the flow resistance in other regions, thereby promoting more uniform water distribution.

In regions where maintaining maximum skin contact is paramount, such as over large flat areas of the torso or thighs, the internal weld points 77 may be arranged in a pattern that maximizes contact area while still permitting adequate water flow. For example, the internal weld points 77 may be arranged in a staggered or offset pattern rather than a grid pattern, to avoid creating linear barriers to water flow while still constraining expansion of the water bag 74.

The goal of the variable spacing of the internal weld points 77 is to maintain substantially balanced pressures throughout the water bag 74, thereby avoiding flow interruptions, flow cuts, or reflux conditions that could result in uneven thermal coverage. By tuning the spacing of the internal weld points 77 by body region, the water bag 74 provides consistent water flow and thermal coverage across all areas of the user's body, regardless of body position or movement.

In one exemplary embodiment, the spacing between adjacent internal weld points 77 in high-bending regions such as the knees may be in the range of approximately 20 mm to 40 mm, while the spacing between adjacent internal weld points 77 in lower-bending regions such as the torso may be in the range of approximately 10 mm to 25 mm. It will be appreciated that these ranges are exemplary only, and the specific spacing may be varied depending on the material properties of the water bag 74, the expected water pressure and flow rate, and the desired flexibility and thermal performance characteristics.

The internal weld points 77 (or any of the welds in the garment) may be formed by any suitable welding technique, including but not limited to heat welding, ultrasonic welding, radio frequency (RF) welding, or adhesive bonding. The internal weld points 77 are preferably circular or substantially circular in shape, although other shapes such as oval, square, or irregular shapes may be employed. The diameter or size of each internal weld point 77 is preferably small relative to the spacing between adjacent weld points, such that the weld points 77 do not substantially occlude water flow within the channels 76.

### Layer Assembly and Outer Weld Points

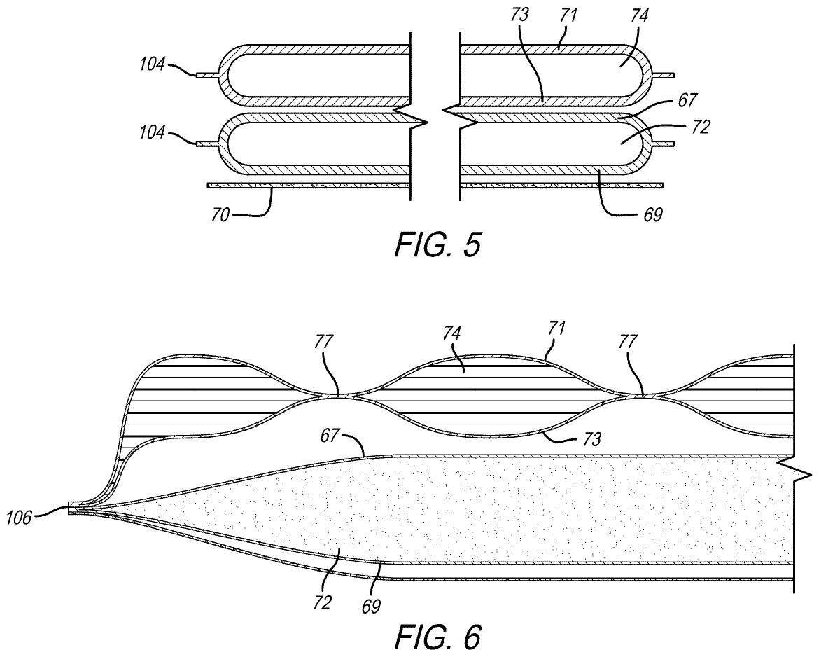

Referring now to FIGS. 5 and 6, there are shown schematic cross-sectional views of the garment layers prior to and after assembly. FIG. 5 illustrates the water bag 74, air bag 72, and outer fabric layer 70 in an unassembled state, prior to being welded together. FIG. 6 illustrates the water bag 74, air bag 72, and outer fabric layer 70 after being welded together at one end 104 and including a plurality of intermittent weld points 108.

As shown in FIG. 6, the layers of each garment portion are joined together at the peripheral edges, such as at the end 104, to form a unified structure. The ends may also be considered tabs 104 (see FIG. 4A). Additionally, the air bag 72 and the water bag 74 are joined together at intermittent weld points 106 disposed at spaced intervals across the surface area of the garment portion. The intermittent weld points 106 serve to couple the air bag 72 to the water bag 74 such that, when the air bag 72 is inflated, the expansion of the air bag 72 presses the water bag 74 toward the user's body.

Critically, the intermittent spacing of the weld points 106 ensures that the air bag 72 can expand without wrinkling or constricting the water channels 76 in the water bag 74. If the air bag 72 and water bag 74 were continuously welded together along their entire interface, expansion of the air bag 72 could cause bunching, folding, or occlusion of the water channels 76, thereby restricting water flow and reducing the effectiveness of the thermal therapy. The intermittent weld points 106 permit the air bag 72 to expand more uniformly and to press the water bag 74 into contact with the user's skin without substantially impeding water flow through the water channels 76.

### Water Flow Routing.

Referring now to FIG. 7, there is shown a plan view of the water bags 74 connected to the cooling and heating unit 14 via hoses and illustrating the water flow path through the garment 60. As shown, the water path extends from the cooling and heating unit 14, through the water inlet hoses 80, 82, through the water channels 76 configured as water highways, through the water outlet hoses 84, 86, and back to the cooling and heating unit 14, thereby forming a closed-loop circulation path.

The water inlet hoses 80, 82 connect to the garment 60 at shoulder inlet connections 90 positioned proximate the user's shoulders. The water outlet hoses 84, 86 connect to the garment 60 at foot outlet connections 92 positioned proximate the user's feet. This shoulder-to-foot flow direction is purposefully selected to deliver the coldest water to the torso region, where the physiological effect is greatest. The coldest water entering the garment 60 at the shoulders extracts heat from the user's core region and flows downwardly, warming as it progresses, and exits at the feet where it returns to the cooling and heating unit 14 for re-conditioning.

In the illustrated embodiment, the water flow path through the water channels 76 bifurcates at the shoulder inlet connections 90 to provide separate flow paths to the arm water bags 74a, 74c and the body water bags 74b, 74d. The arm water bags 74a, 74c may be configured as loop paths that return to the body water bags 74b, 74d at the shoulders, or may include separate outlet connections at the hands or wrists. In the preferred embodiment, the arm water bags 74a, 74c include the channels 76 that are configured as loop paths such that water entering the arm from the shoulder flows through the arm water bag, around the arm, and returns to the body water bag to continue flowing toward the feet. It will be appreciated that there are two water inlets, one per side of the body. Each of the inlets bifurcate to feed the four channels 76 per side (total 8 channels in the two body water bags 74b and 74d), as shown in FIG. 7.

The body water bags 74b, 74d each define four substantially parallel water channels 76 extending from the shoulder region to the foot region. The four channels provide sufficient flow capacity to deliver adequate water volume to all areas of the user's torso and legs. The channels are separated by weld lines 78 that extend substantially vertically when the garment 60 is worn, minimizing horizontal flow and ensuring rapid, direct transit of temperature-conditioned water.

### Garment Inflation and Body Contact

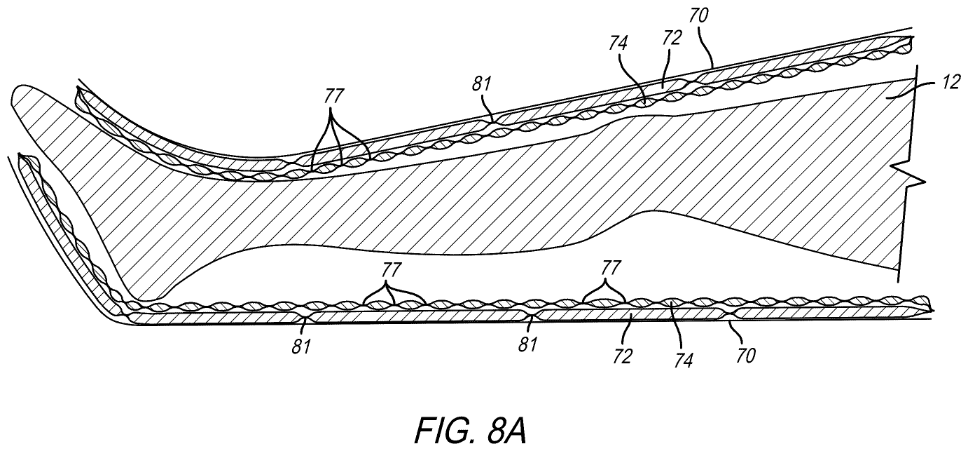

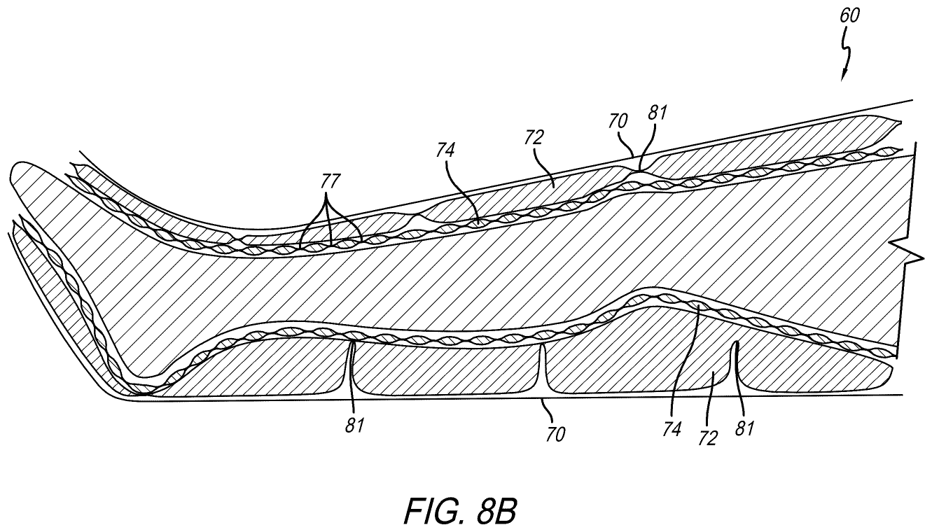

Referring now to FIGS. 8A and 8B, there are shown side cross-sectional views of a user's leg 12 in a leg section of the garment 60 prior to and after inflation of the air bag 72, respectively. It will be appreciated that in both FIGS. 8A and 8B, the cross-section is taken along lines that show the weld lines 81 or weld points 77. These figures illustrate the function of the air bag 72 in pressing the water bag 74 into uniform contact with the user's skin.

As shown in FIG. 8A, prior to inflation, the water bag 74 may not be in complete contact with the user's leg 12. Gaps or spaces may exist between the water bag 74 and the user's skin, particularly in areas where the leg 12 has irregular contours, such as the back of the knee or the calf. These gaps reduce the effectiveness of thermal transfer between the temperature-conditioned water in the water bag 74 and the user's skin.

As shown in FIG. 8B, after inflation of the air bag 72, the air bag 72 expands to fill the space between the outer fabric layer 70 and the water bag 74, thereby pressing the water bag 74 into intimate contact with the user's leg 12. The inflated air bag 72 conforms to the irregular contours of the user's leg 12 and presses the water bag 74 into contact with substantially the entire surface of the leg 12. This uniform contact ensures effective thermal transfer between the temperature-conditioned water and the user's skin across all areas of the garment 60.

The inflation pressure of the air bag 72 is preferably controlled by the cooling and heating unit 14 to achieve optimal body contact without causing discomfort to the user. In some embodiments, the inflation pressure may be varied across different regions of the garment 60 to achieve a pressure gradient, such as higher pressure at the feet and lower pressure at the torso, to simulate the hydrostatic pressure gradient experienced when standing in water. In some embodiments, the air bag 72 may be divided into a plurality of independently inflatable zones, each zone having a separate air supply line or controllable valve, to enable differential pressure across different body regions.

### Cooling and Heating Unit Construction

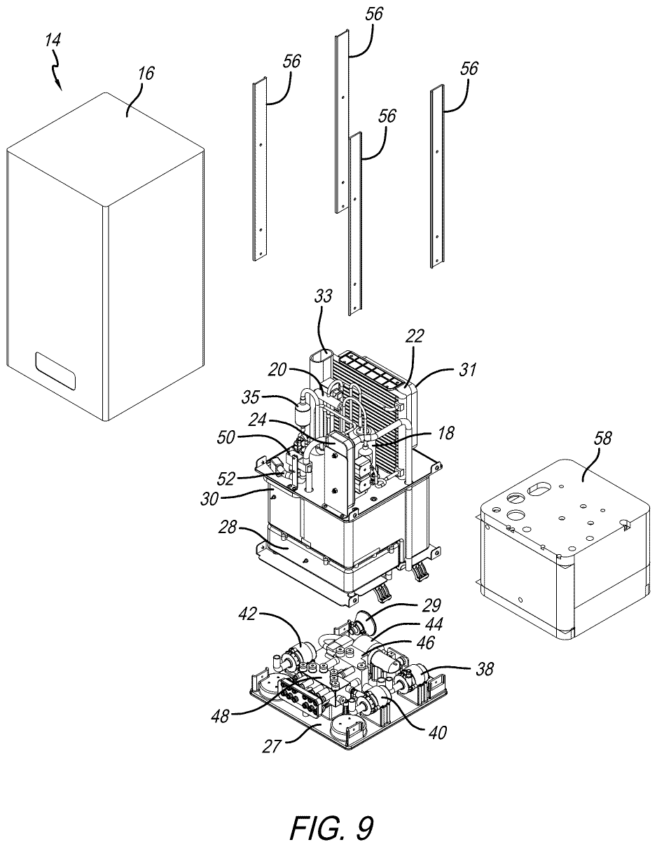

Referring now to FIG. 9, there is shown a partial exploded view of the cooling and heating unit 14. The cooling and heating unit 14 comprises a housing 16 that encloses the various components of the unit. The housing 16 is preferably compact and portable, enabling the cooling and heating unit 14 to be positioned adjacent to a bed, treatment table, or other surface on which a user lies while wearing the garment 60.

The cooling and heating unit 14 includes a refrigerant loop comprising a compressor 18, a four-way reversing valve 20, a heat sink 22, and a plate heat exchanger 24. The compressor 18 compresses refrigerant gas, which then flows through the four-way reversing valve 20, through the heat sink 22, through a first capillary tube 52, through the plate heat exchanger 24, and returns to the compressor 18, thereby completing the refrigerant loop. The four-way reversing valve 20 enables the direction of refrigerant flow to be reversed, thereby switching the cooling and heating unit 14 between a cooling mode and a heating mode. In the cooling mode, the plate heat exchanger 24 functions as an evaporator and extracts heat from water circulating therethrough. In the heating mode, the plate heat exchanger 24 functions as a condenser and delivers heat to water circulating therethrough.

The cooling and heating unit 14 further includes a water tank system comprising a large water tank 26 and a small water tank 28. The large water tank 26 and small water tank 28 are fluidly connected through a restricted interconnection 36. The restricted interconnection 36 has a flow area that is substantially smaller than the cross-sectional areas of the tanks 26, 28, thereby limiting the rate of water migration between the tanks. This restricted interconnection 36 enables the system to confine heating or cooling effects to one tank while limiting the mixing of water between the tanks.

The small water tank 28 includes a one-way divider 34 disposed therein. The one-way divider 34 is configured to permit water flow in a first direction and to substantially prevent water flow in an opposite direction. In one embodiment, the one-way divider 34 comprises a pivoting trapdoor that opens under flow pressure in the first direction and closes under flow pressure in the opposite direction. The one-way divider 34 prevents backflow or back-mixing of water from a return side of the small water tank 28 to a supply side of the small water tank 28, thereby enabling the system to maintain temperature separation within the small water tank 28 during heating or cooling operations.

The cooling and heating unit 14 further includes an ice-making tank 30 that is preferably formed as a subdivision or extension of the large water tank 26. The ice-making tank 30 includes an ice-making coil 32 disposed therein. The ice-making coil 32 is preferably a helical or serpentine metal tube through which refrigerant flows. The ice-making coil 32 is in thermal communication with the refrigerant loop through a dedicated capillary branch including a second capillary tube 54. When the refrigerant loop is configured to direct refrigerant through the second capillary tube 54 and the ice-making coil 32 while water in the ice-making tank 30 is substantially static, the ice-making coil 32 extracts heat from the surrounding water, causing ice to form around the ice-making coil 32. The ice thus formed serves as a thermal battery that can subsequently be used to cool water circulating through the ice-making tank 30.

The cooling and heating unit 14 includes a plurality of water pumps for circulating water through the system. An internal circulation pump (or water pump one) 38 is configured to circulate water between the water tank system and the plate heat exchanger 24 in a closed internal loop. This internal circulation enables the cooling and heating unit 14 to pre-cool or pre-heat water in the tank system before any water is circulated to the garment 60, thereby ensuring that the user experiences an immediate thermal effect upon commencement of garment circulation. A first garment pump (or water pump two) 40 and a second garment pump (or water pump three) 42 are configured to circulate water through the garment 60. In the preferred embodiment, the first garment pump 40 supplies water to the left body portion 64 and left arm sleeve 68, while the second garment pump 42 supplies water to the right body portion 62 and right arm sleeve 66. This dual-pump arrangement reduces the flow path length for each pump and maintains adequate flow rates to all portions of the garment 60.

The cooling and heating unit 14 further includes an air pump 44 for inflating the air bags 72 in the garment 60. The air pump 44 is fluidly connected to the garment 60 through the air hose 88. The air pump 44 is configured to supply pressurized air to the air bags 72 at a controlled flow rate and pressure to achieve the desired inflation level.

The cooling and heating unit 14 includes one or more water valve blocks 46, 48 that integrate multiple valves for controlling water flow through the system. The water valve blocks 46, 48 are preferably positioned proximate the hose connections to the garment 60 to minimize flow path lengths and reduce pressure losses. The valves within the water valve blocks 46, 48 enable the cooling and heating unit 14 to selectively direct water flow to different portions of the system depending on the operating mode.

The cooling and heating unit 14 further includes a three-way purge valve (or three way valve 1) 50 that selectively connects the air pump 44 to the water channels in the garment 60. When the three-way purge valve 50 is actuated, pressurized air from the air pump 44 is directed into the water inlet hoses 80, 82, thereby forcing any residual water remaining in the water bags 74 back through the water outlet hoses 84, 86 and into the water tank system. This purge function enables the garment 60 to be substantially emptied of water at the end of a therapy session, reducing the weight of the garment 60, returning water to the cooling and heating unit 14 for re-conditioning, and ensuring that the next therapy session begins with freshly conditioned water at the garment inlets.

The pumps 38, 40, 42 are preferably positioned outside of the water tanks 26, 28, 30 and at or below the lowest water level in the tank system. This positioning improves pump efficiency by ensuring that the pumps operate with positive suction head and minimizes heat transfer from the pumps to the water in the tanks.

The cooling and heating unit 14 further includes a frame or series of brackets 56 for supporting the various components within the housing 16, and insulation 58 surrounding the water tanks 26, 28, 30 and other components to minimize heat transfer between the cooling and heating unit 14 and the ambient environment. The cooling and heating unit 14 may also include a bottom housing 27, drain hole 29, fan 31, water inlet 33 and one or more liquid storage tanks 35, among other components.

### System Flow Diagram and Operational Modes

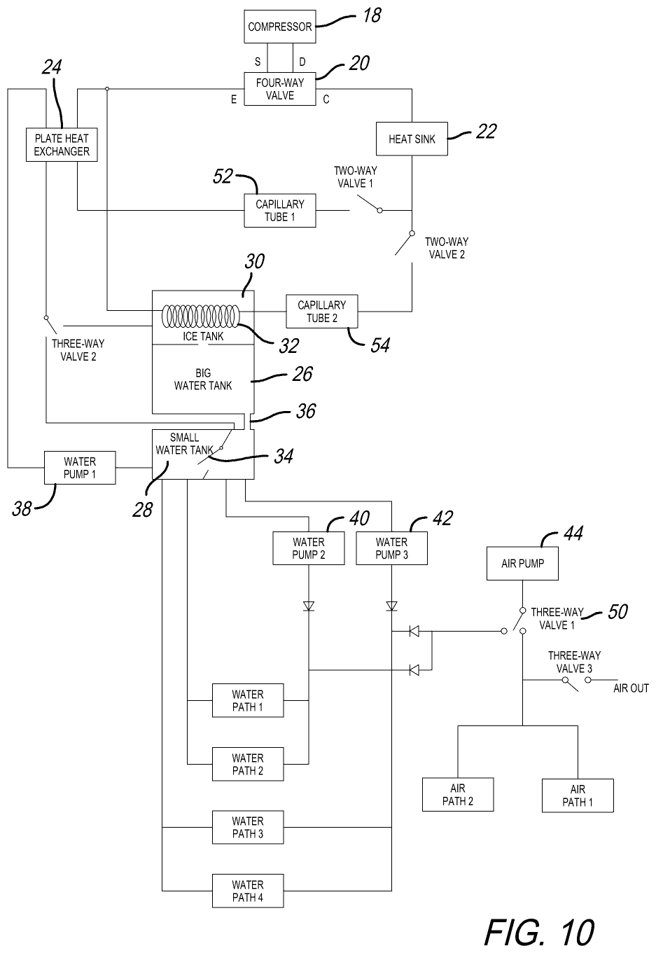

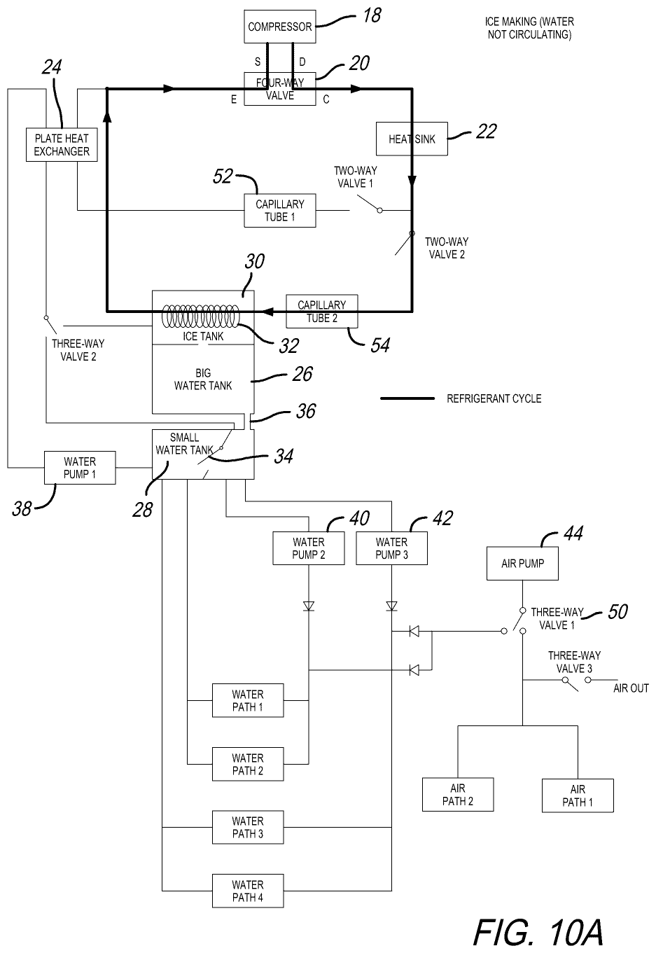

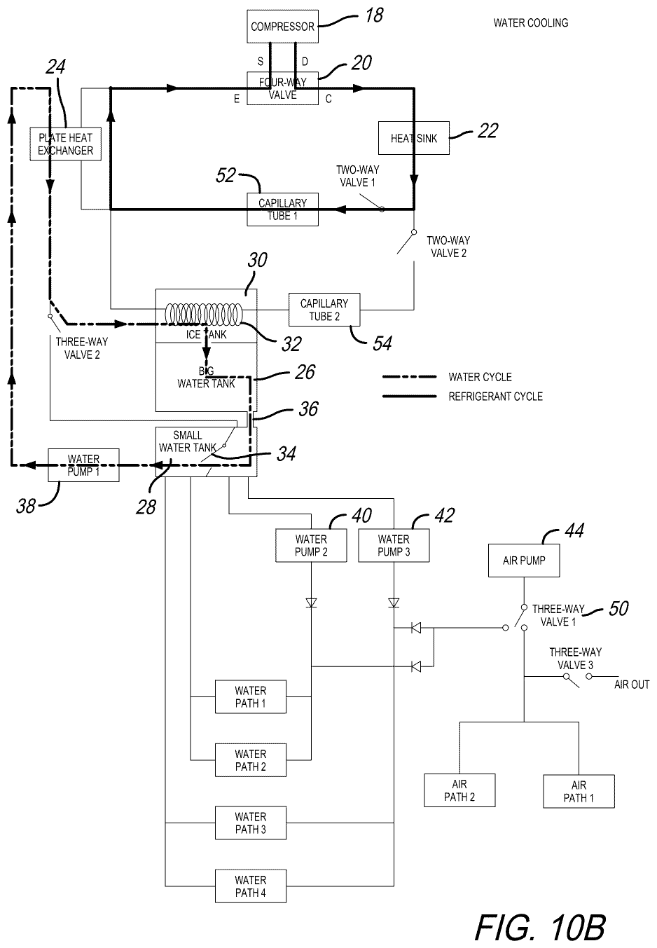

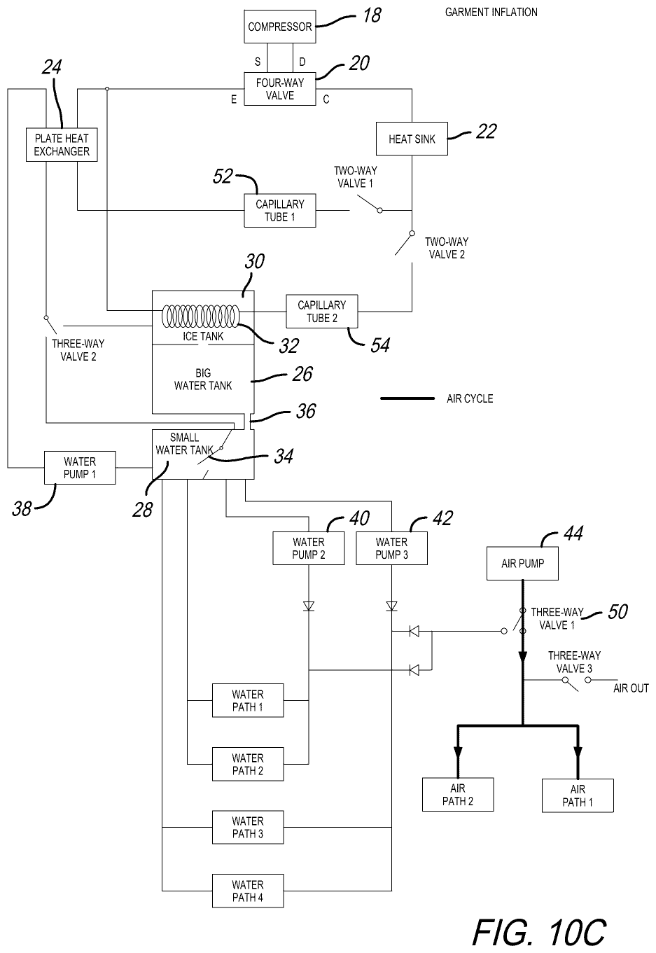

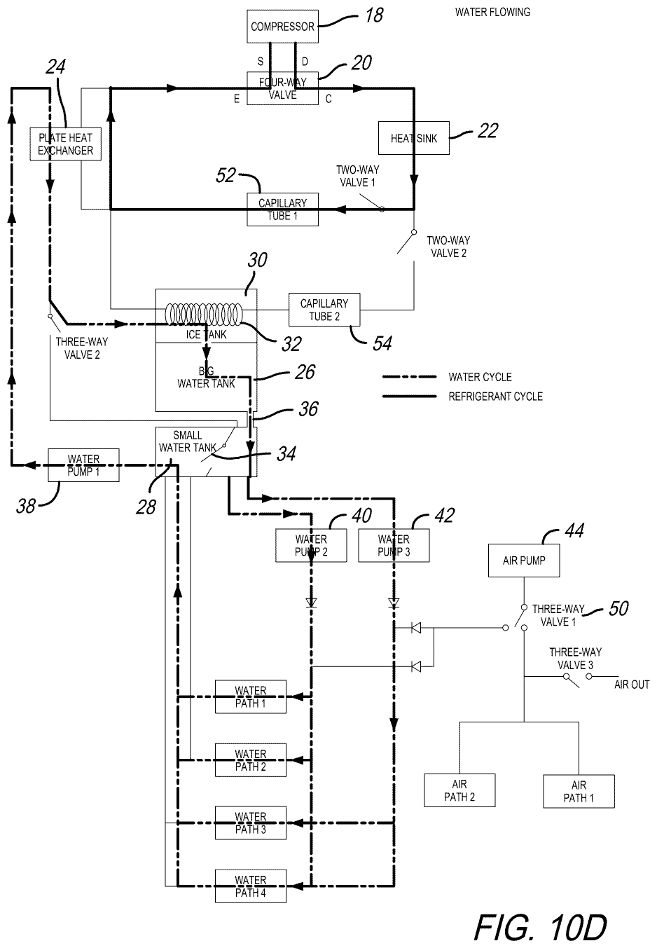

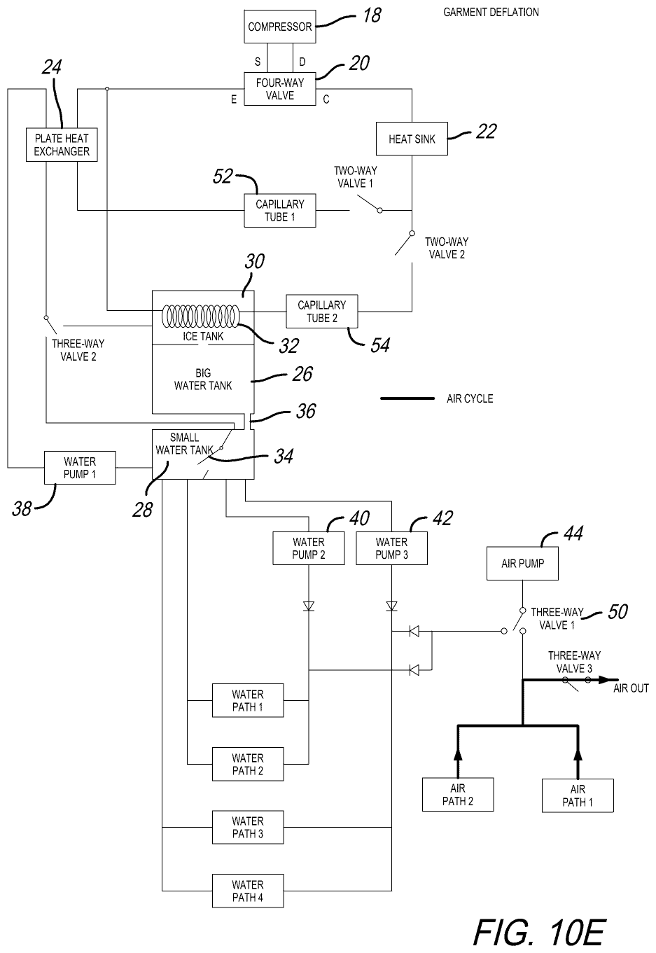

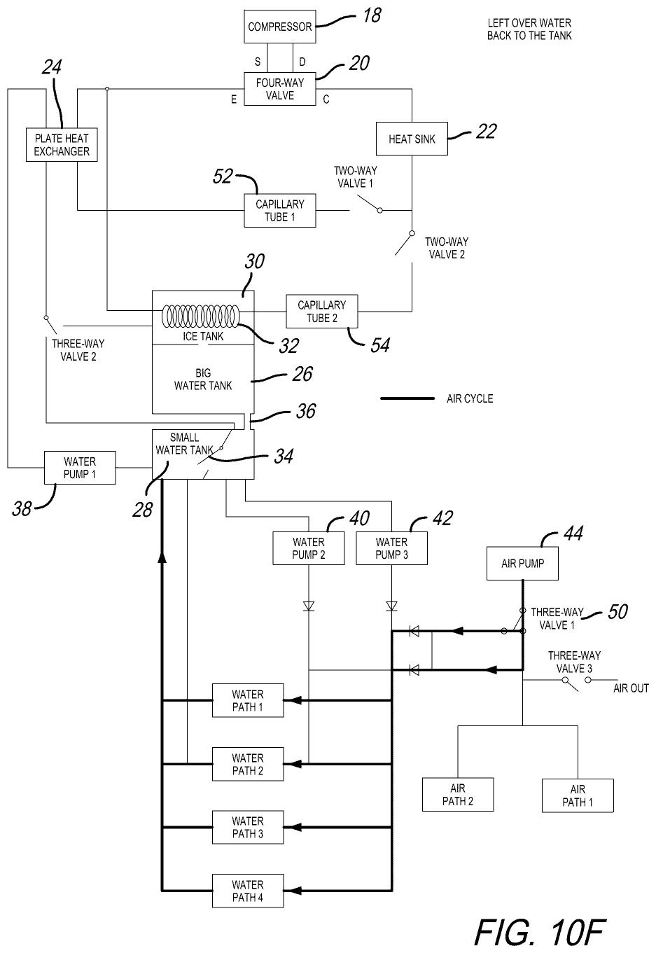

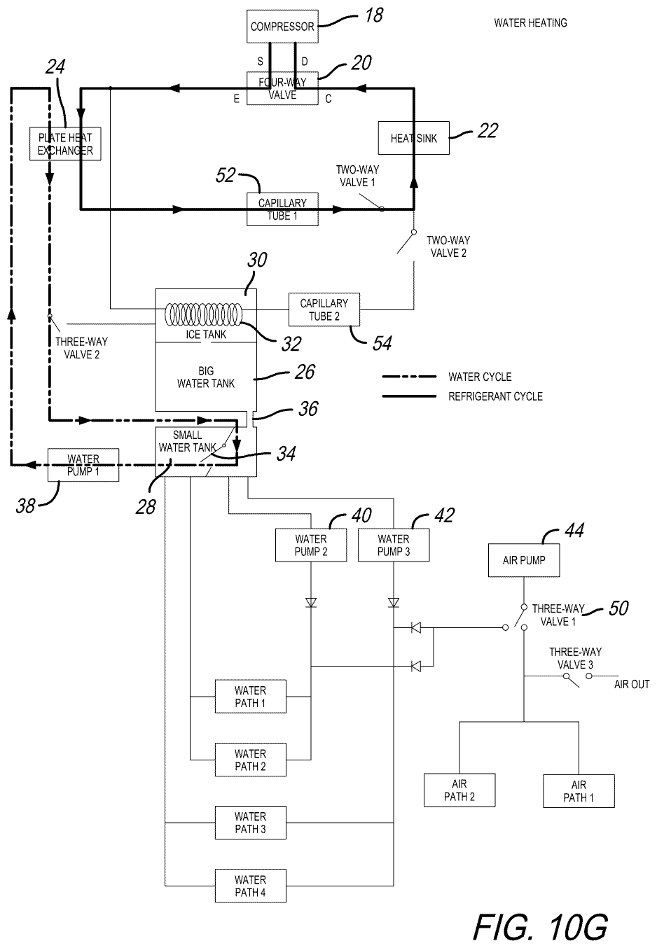

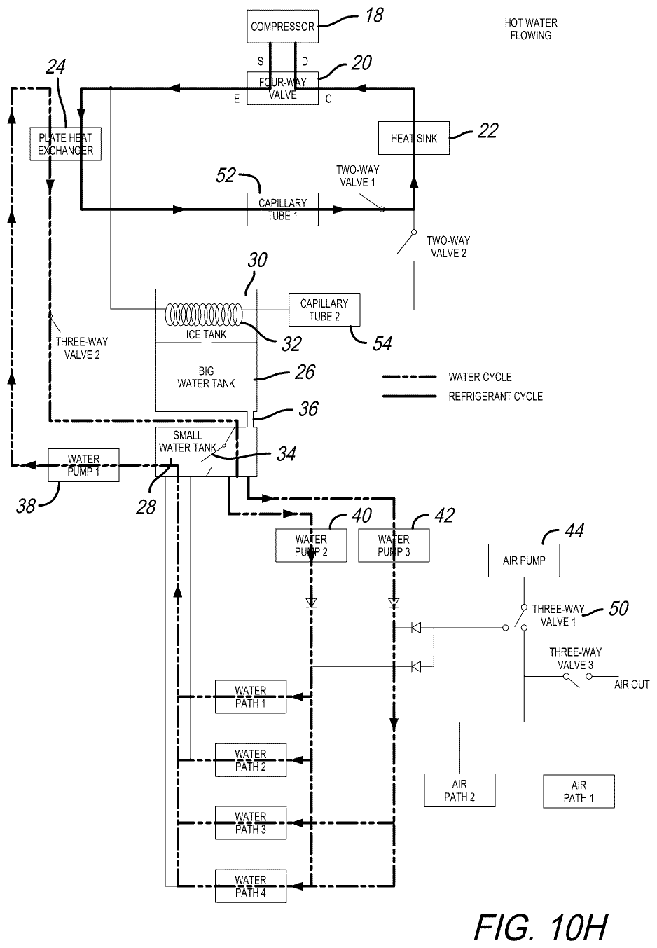

Referring now to FIGS. 10 and 10A-10H, there are shown a system flow diagram and a series of diagrams illustrating the various operational modes of the thermal therapy system 10. FIG. 10 is a flow diagram of the system showing the refrigerant loop components, water tank system, pumps, valves, and connections to the garment 60. FIGS. 10A-10H illustrate the refrigerant cycle, water cycle, and/or air cycle during each of the following operational modes: ice making (FIG. 10A), water cooling (FIG. 10B), garment inflation (FIG. 10C), cool water flow (FIG. 10D), garment deflation (FIG. 10E), water return/purge (FIG. 10F), water heating (FIG. 10G), and hot water flow (FIG. 10H).

### Ice Making Mode

Referring to FIG. 10A, the ice making mode is illustrated. In the ice making mode, the refrigerant loop is configured to direct refrigerant through the ice-making coil 32 in the ice-making tank 30. The four-way reversing valve 20 is set to the cooling mode orientation, and a valve associated with the second capillary tube 54 is opened to permit refrigerant flow through the ice-making coil 32. The compressor 18 compresses refrigerant, which flows through the four-way reversing valve 20, releases heat at the heat sink 22, expands through the second capillary tube 54, extracts heat from the ice-making coil 32, and returns to the compressor 18.

During the ice making mode, water in the ice-making tank 30 is maintained substantially static, i.e., the internal circulation pump 38 and the garment pumps 40, 42 are not circulating water through the ice-making tank 30. The static water surrounding the ice-making coil 32 enables efficient ice formation, as the cold surface of the coil 32 extracts heat from the immediately adjacent water without the warming effect of flowing water. Ice progressively forms around the ice-making coil 32, building a block of ice that serves as a thermal battery for subsequent cooling operations.

The ice making mode is preferably performed prior to or concurrent with the initial water cooling operations, such that a substantial quantity of ice is formed in the ice-making tank 30 before water circulation through the garment 60 is commenced. In one embodiment, the ice making mode is performed during a pre-conditioning period while the user is donning the garment 60 and before the therapy session begins.

Referring again to FIGS. 10 and 10A, the ice-making tank 30 and ice-making coil 32 provide a key differentiating feature of the thermal therapy system 10. The ice-making coil 32, which is preferably a helical metal tube through which refrigerant flows, is disposed within a region of the water tank system. During the ice-making mode, when water in this region is substantially static, the ice-making coil 32 extracts heat from the surrounding water and causes ice to form around the coil 32. The ice thus formed serves as a thermal battery for subsequent cooling operations. What is particularly significant about this design is not merely the presence of a separate ice-making tank, but rather the system's ability to force return water from the garment 60 to flow through and contact the ice formed around the ice-making coil 32 before the water continues to the main water tank system. This forced routing ensures that heat is extracted from the return water by the ice, thereby maintaining a lower and more stable water temperature throughout the therapy session. The ice-making coil 32 may be disposed within a separate ice-making tank 30, or the ice-making coil 32 may be disposed within a region of a larger unified tank with baffles or flow guides that direct return water through the region containing the ice. In either configuration, the key functional feature is the forced contact between return water and the ice formed around the ice-making coil 32, which leverages the latent heat of fusion of the ice to provide sustained cooling capacity beyond what the plate heat exchanger 24 alone could provide. The ice making preferably occurs after the water has been cooled. However, in another embodiment, the ice may be made before the water is cooled. It will be appreciated that having a separate tank is not a requirement and a divider is shown between the main tank and the ice making tank to show that the water is forced to go through the ice block in this embodiment.

### Water Cooling Mode

Referring to FIG. 10B, the water cooling mode is illustrated. In the water cooling mode, the refrigerant loop is configured to direct refrigerant through the plate heat exchanger 24, and the internal circulation pump 38 circulates water between the water tank system and the plate heat exchanger 24 in a closed internal loop. The compressor 18 compresses refrigerant, which flows through the four-way reversing valve 20, releases heat at the heat sink 22, expands through the first capillary tube 52, extracts heat from water in the plate heat exchanger 24, and returns to the compressor 18. The internal circulation pump 38 draws water from the water tank system, pumps it through the plate heat exchanger 24 where the water is cooled, and returns the cooled water to the water tank system.

During the water cooling mode, the garment pumps 40, 42 are preferably not operating, and water is not circulating through the garment 60. This closed internal loop enables the cooling and heating unit 14 to pre-cool the water in the tank system to a desired setpoint temperature before any water is circulated to the garment 60. In one embodiment, the water is cooled to a setpoint temperature of approximately 2° C., which, when circulated through the garment 60, produces a skin temperature experience similar to immersion in a cold water bath at approximately 10° C. The pre-cooling ensures that the user experiences an immediate “cold shock” sensation upon commencement of water circulation through the garment 60. This may provide a correlation between the water temperature and the equivalent skin temperature in a traditional cold plunge.

### Garment Inflation Mode

Referring to FIG. 10C, the garment inflation mode is illustrated. In the garment inflation mode, the air pump 44 supplies pressurized air through the air hose 88 to the air bags 72 in the garment 60. The three-way purge valve 50 is positioned to connect the air pump 44 to the air hose 88 rather than to the water inlet hoses 80, 82. As the air pump 44 operates, air pressure builds within the air bags 72, causing the air bags 72 to inflate and expand inwardly toward the user's body.

The garment inflation mode may be performed independently of the water cooling or circulation modes. In one embodiment, the garment inflation mode is performed after the user has donned the garment 60 and before water circulation is commenced, such that the air bags 72 are inflated and the water bags 74 are pressed into contact with the user's skin prior to the introduction of temperature-conditioned water. In another embodiment, the garment inflation mode is performed concurrently with water circulation.

The air pump 44 preferably includes a pressure sensor and controller that monitors the air pressure within the air bags 72 and controls the air pump 44 to maintain the air pressure within a desired range. The controller may enable the user to select a desired inflation pressure or may automatically adjust the inflation pressure based on user feedback or sensor inputs.

### Cool Water Flow Mode

Referring to FIG. 10D, the cool water flow mode is illustrated. In the cool water flow mode, the refrigerant loop continues to operate in the cooling mode, the internal circulation pump 38 continues to circulate water through the plate heat exchanger 24, and the garment pumps 40, 42 circulate water from the water tank system through the garment 60. The first garment pump 40 draws water from the water tank system, pumps it through the water inlet hose 80 to the left body portion 64 and left arm sleeve 68 of the garment 60, receives return water from the water outlet hose 84, and returns the water to the water tank system. The second garment pump 42 similarly circulates water through the right body portion 62 and right arm sleeve 66. When the water returns from the garment 60, it passes through the cooling cycle before going back into the tank. This may be done to maximize cooling efficiency. The greater the temperature difference between the water and the refrigerant, the more efficient the cooling process becomes. Additionally, if the water were sent directly back to the tank, it would raise the overall temperature of the stored water. By routing it through the cooling cycle first, the water is cooled down before re-entering the tank, helping maintain a lower overall temperature.

During the cool water flow mode, the return water from the garment 60 is preferably routed through the ice-making tank 30 before returning to the main water tank system. As the return water passes through the ice-making tank 30, the water contacts the ice block formed around the ice-making coil 32, and heat is extracted from the return water by the melting ice. This ice-assisted cooling enables the system to maintain a lower and more stable water temperature throughout the therapy session than would be possible with the plate heat exchanger 24 alone. The latent heat of fusion of the ice provides a substantial heat sink that stabilizes the water temperature and extends the effective cooling capacity of the system.

The refrigerant loop continues to operate during the cool water flow mode, providing additional cooling capacity through the plate heat exchanger 24. The combination of the plate heat exchanger 24 and the ice-making tank 30 enables the system to deliver and sustain cold water at the garment inlets throughout the therapy session.

### Garment Deflation Mode

Referring to FIG. 10E, the garment deflation mode is illustrated. In the garment deflation mode, the air pump 44 is stopped, and one or more air release valves are opened to permit the pressurized air within the air bags 72 to escape to the ambient environment. In one embodiment, at least one of the air release valves is an emergency air release valve 102 accessible to the user on the garment 60 itself, positioned proximate the user's hips or other accessible location. The emergency air release valve 102 enables the user to rapidly depressurize the air bags 72 in the event of an emergency or discomfort. Various air release valves and input openings (where the air hoses 88 connect) are shown in FIG. 4A (e.g., near the user's feet and shoulders). Furthermore, openings in the body and arm air bags and connection hoses between the body and arm air bags may be provided to allow air to flow from the body air bags to the inflate the arm air bags or vice versa. The air flow path extends between the different garment portions.

During normal operation, the garment deflation mode may be performed at the end of a therapy session, after water circulation has been stopped and before the user doffs the garment 60. The deflation of the air bags 72 releases the pressure holding the water bags 74 against the user's skin and enables the user to move freely within the garment 60.

### Water Return (Purge) Mode

Referring to FIG. 10F, the water return mode (also referred to as the purge mode) is illustrated. In the water return mode, the three-way purge valve 50 is actuated to connect the air pump 44 to the water inlet hoses 80, 82 rather than to the air hose 88. The air pump 44 is then operated to supply pressurized air into the water inlet hoses 80, 82. The pressurized air enters the water bags 74 of the garment 60 through the shoulder inlet connections 90 and forces any residual water remaining in the water channels 76 back through the water outlet hoses 84, 86 and into the water tank system.

The water return mode purges substantially all of the residual water from the water bags 74, leaving the garment 60 substantially dry and light. This purge function provides several advantages. First, the reduced water weight makes the garment 60 easier to handle and store between therapy sessions. Second, the returned water is collected in the water tank system where it can be re-conditioned for the next therapy session. Third, the purge ensures that the next therapy session begins with freshly conditioned water at the garment inlets, rather than stale water that has been sitting in the garment 60 since the previous session. One of the benefits of pushing all the water back into the tank after a session is that the it allows the water temperature to be lower. The water that stays in the garment will not be cooled or heated and therefore affect the performance of the session.

### Water Heating Mode

Referring to FIG. 10G, the water heating mode is illustrated. In the water heating mode, the four-way reversing valve 20 is set to the heating mode orientation, reversing the direction of refrigerant flow through the refrigerant loop. The compressor 18 compresses refrigerant, which flows through the four-way reversing valve 20, releases heat at the plate heat exchanger 24 (now functioning as a condenser), expands through the capillary tube, extracts heat from the ambient environment at the heat sink 22 (now functioning as an evaporator), and returns to the compressor 18. The internal circulation pump 38 circulates water between the water tank system and the plate heat exchanger 24, where the water is heated by the refrigerant.

During the water heating mode, the water flow is preferably directed through the small water tank 28 rather than the large water tank 26. The restricted interconnection 36 between the large water tank 26 and the small water tank 28 limits the migration of water between the tanks, such that the heating effect is substantially confined to the water in the small water tank 28. This enables the system to rapidly heat a smaller volume of water (e.g., approximately one-third of the total water volume, such as approximately 4 liters out of a total volume of approximately 12 liters) while maintaining the larger volume of water in the large water tank 26 at a cooler temperature.

The one-way divider 34 in the small water tank 28 prevents backflow or back-mixing of the heated return water into the supply side of the small water tank 28, thereby maintaining temperature separation within the small water tank 28. This configuration enables rapid heating to a desired hot water setpoint and enables rapid transitions back to the cooling mode by drawing upon the cold water reservoir maintained in the large water tank 26.

### Hot Water Flow Mode

Referring to FIG. 10H, the hot water flow mode is illustrated. In the hot water flow mode, the refrigerant loop continues to operate in the heating mode, the internal circulation pump 38 continues to circulate water through the plate heat exchanger 24, and the garment pumps 40, 42 circulate heated water from the small water tank 28 through the garment 60. The water flow path during the hot water flow mode is substantially similar to the cool water flow mode, except that the water is drawn preferentially from the small water tank 28 rather than the large water tank 26, and the heated water delivers heat to the user's body rather than extracting heat.

During the hot water flow mode, the one-way divider 34 prevents the heated return water from mixing with the supply water in the small water tank 28, maintaining the temperature differential between the supply and return sides of the tank. This configuration enables the system to deliver a consistent heated water temperature to the garment 60 throughout the therapy session.

### Garment Closure System

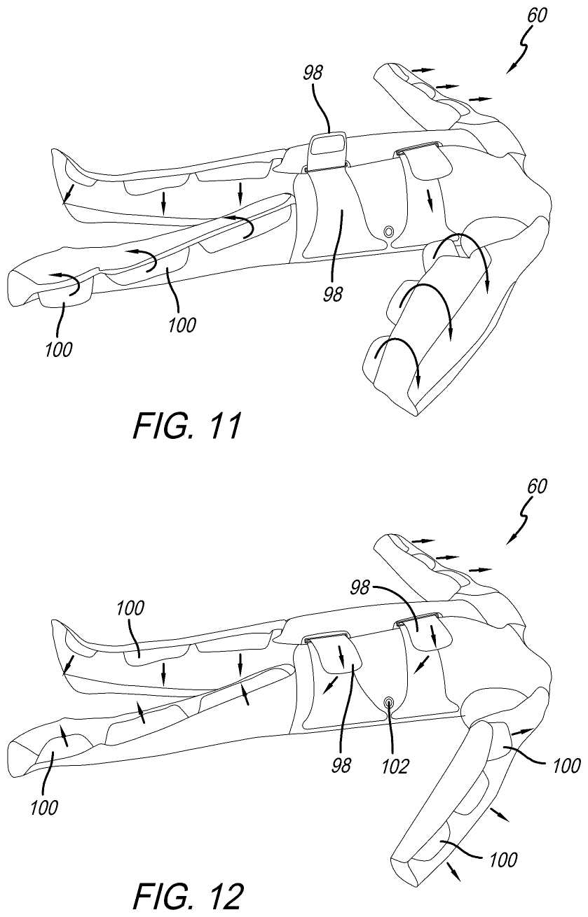

Referring now to FIGS. 11 and 12, there are shown perspective views of the garment 60 with the straps 98 and tabs 100 in open and closed positions, respectively. The garment 60 includes a plurality of closures for securing the garment 60 around the user's body when the garment 60 is in the closed position.

As shown in FIG. 11, the garment 60 is depicted with some of the straps 98 and tabs 100 in the open position. The straps 98 are elongated members that extend from one portion of the garment 60 to another and are secured by fasteners such as hook-and-loop fasteners (e.g., Velcro®), snaps, buckles, or other suitable closure mechanisms. The tabs 100 are smaller closure elements that secure adjacent edges of the garment 60 together.

As shown in FIG. 12, the garment 60 is depicted with all straps 98 and tabs 100 in the closed position. In this configuration, the garment 60 is fully closed around the user's body, and the air bags 72 and water bags 74 are positioned for optimal thermal contact with the user's skin.

The closures are preferably arranged to enable the user to don and doff the garment 60 without assistance. In one embodiment, the closures include leg closures 100 extending along the inner leg seam from the ankle to the crotch, and chest closures or straps 98 extending along the front of the garment 60 from the waist to the neck. The leg closures 100 enable the user to open the leg portions of the garment 60 to insert the legs, and the chest closures 98 enable the user to wrap the body portions around the torso and secure them in place.

The closures preferably comprise hook-and-loop fasteners that provide secure closure without gaps that could interfere with thermal contact or permit leakage of air from the air bags 72. The hook-and-loop fasteners are preferably arranged with overlapping flaps that seal the closure and provide a smooth interior surface against the user's skin.

The fully openable construction of the garment 60 also facilitates cleaning. The user or operator can open the garment 60 fully to expose all interior surfaces, enabling thorough cleaning and sanitization between users. This is particularly advantageous for commercial applications in which the garment 60 may be used by multiple users.

### Remote Controller

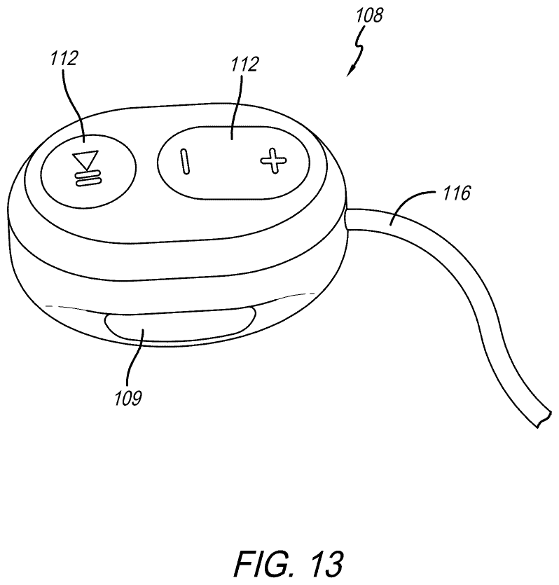

Referring now to FIG. 13, there is shown a perspective view of a remote controller 108 for the thermal therapy system 10. The remote controller 108 enables the user to control the operation of the thermal therapy system 10 from a convenient location, such as on the user's wrist while wearing the garment 60.

The remote controller 108 may include a display for presenting information to the user, such as current water temperature, air pressure, elapsed session time, and biometric data. The remote controller 108 further includes one or more control buttons 112 for enabling the user to select operating modes, adjust temperature setpoints, start and stop water circulation, and perform other control functions.

In the preferred embodiment, the remote controller 108 is connected to the cooling and heating unit 14 through a controller wire 116 that may be routed through one of the hoses connecting the cooling and heating unit 14 to the garment 60 or may be routed separately. The controller wire 116 emerges from the garment at a location proximate the user's wrist or forearm, enabling the user to access the remote controller 108 while wearing the garment 60 without removing the hands from the garment 60. The controller 108 may include a finger opening 109 for a user to place the controller on their finger.

The remote controller 108 further includes a biometric sensor for sensing one or more biometric parameters of the user. In one embodiment, the biometric sensor comprises a photoplethysmography (PPG) sensor for sensing the user's heart rate. The biometric sensor may also be configured to sense other biometric parameters, such as heart rate variability (HRV), respiratory rate, blood oxygen saturation (SpO2), and skin temperature. The biometric parameters sensed by the biometric sensor may be used to infer additional physiological parameters, such as core body temperature.

The thermal therapy system 10 may be configured to operate in a closed-loop control mode responsive to the biometric parameters sensed by the biometric sensor. For example, the system may be configured to automatically adjust the water temperature or session duration based on the user's heart rate response. In one embodiment, the system monitors the user's heart rate and adjusts the water temperature to maintain the heart rate within a target range, providing a safe and effective thermal therapy session. In another embodiment, the system monitors the user's heart rate variability and terminates the session or transitions to a warming mode when certain HRV thresholds are reached.

The closed-loop control mode provides several advantages over open-loop control. First, the system can adapt to individual user physiology, providing a personalized therapy experience. Second, the system can provide safety monitoring to prevent adverse physiological responses to extreme temperatures. Third, the system can optimize the therapy session based on objective physiological data rather than subjective user feedback.

The remote controller 108 may further include a wrist strap or other attachment mechanism for securing the remote controller 108 to the user's wrist or forearm during operation. The wrist strap ensures that the remote controller 108 remains accessible to the user throughout the therapy session.

### Additional Features and Embodiments

The thermal therapy system 10 may include various additional features and alternative embodiments within the scope of the present invention.

In some embodiments, the garment 60 may include emergency air release valves 102 positioned at accessible locations on the garment 60, such as proximate the user's hips. The emergency air release valves 102 enable the user to rapidly depressurize the air bags 72 in the event of an emergency, without requiring access to the cooling and heating unit 14 or the remote controller 108.

In some embodiments, the cooling and heating unit 14 may be configured to generate a pressure gradient across the air bags 72 of the garment 60. For example, the system may be configured to inflate the air bags 72 at the feet to a higher pressure than the air bags 72 at the torso, thereby simulating the hydrostatic pressure gradient experienced when standing in water. This pressure gradient may provide therapeutic benefits similar to pneumatic compression therapy while simultaneously providing thermal therapy.

In some embodiments, the air bags 72 may be divided into a plurality of independently inflatable zones, each zone having a separate air supply line or controllable valve. The zones may correspond to different body regions, such as the feet, lower legs, upper legs, torso, and arms. The cooling and heating unit 14 may be configured to independently control the inflation pressure of each zone to achieve a desired pressure distribution across the user's body.

In some embodiments, the water channels 76 may include flow balancing features, such as orifices, restrictors, or valves, that ensure substantially uniform water flow across all channels despite differences in channel length or flow resistance. The flow balancing features may be positioned at the shoulder inlet connections 90, at the foot outlet connections 92, or at intermediate locations along the water channels 76.

In some embodiments, the shoulder inlet connections 90 and/or foot outlet connections 92 may include connector geometries that minimize kinking or occlusion of the water flow path. For example, the connections may include L-shaped or curved fittings that redirect the hose direction without sharp bends that could restrict water flow.

In some embodiments, the cooling and heating unit 14 may include sensors for monitoring system parameters, such as water temperature at various locations in the system, water flow rate, air pressure in the air bags 72, and refrigerant pressure and temperature. The sensor data may be displayed on the remote controller 108 and/or used by the control system to optimize system operation and detect fault conditions. The remote may have a display. The cooling unit may also include a display. In a preferred embodiment, the system may be controlled and programmed via a software application or app. This may be particularly convenient if the user desires to ready the cooling unit when they are remote, e.g., coming home from work, and they want the device to be ready by the time they get home.

In some embodiments, the cooling and heating unit 14 may include a data logging function that records system parameters and biometric data throughout each therapy session. The logged data may be stored in memory within the cooling and heating unit 14 or the remote controller 108, and may be exported to an external device for analysis.

### Methods of Operation

The thermal therapy system 10 may be operated in accordance with various methods within the scope of the present invention. In a method of operating the thermal therapy system 10 for cold therapy, the method including one or more or any of the steps of: donning the garment 60; activating the ice making mode to form ice in the ice-making tank 30; activating the water cooling mode to pre-cool water in the tank system; activating the garment inflation mode to inflate the air bags 72 and press the water bags 74 into contact with the user's body; activating the cool water flow mode to circulate cold water through the garment 60, routing return water through the ice-making tank 30 to extract additional heat; and, upon completion of the therapy session, activating the garment deflation mode and the water return mode to deflate the air bags 72 and purge residual water from the water bags 74.

In a method of operating the thermal therapy system 10 for hot therapy, the method may include one or more or any of the steps of: donning the garment 60; activating the water heating mode to heat water in the small water tank 28; activating the garment inflation mode to inflate the air bags 72 and press the water bags 74 into contact with the user's body; activating the hot water flow mode to circulate hot water through the garment 60; and, upon completion of the therapy session, activating the garment deflation mode and the water return mode to deflate the air bags 72 and purge residual water from the water bags 74.

In a method of operating the thermal therapy system 10 for contrast therapy, the method includes alternating between cold therapy and hot therapy. The method may include one or more or any of the steps of the steps of: performing cold therapy for a first period; transitioning to the water heating mode to heat water in the small water tank 28 while the large water tank 26 remains cold; performing hot therapy for a second period; transitioning back to the cool water flow mode by drawing upon the cold water reservoir in the large water tank 26; and repeating the alternating cold and hot therapy as desired. The two-tank architecture with the one-way divider 34 and restricted interconnection 36 enables rapid transitions between cold and hot therapy by confining the heating effect to the small water tank 28 while preserving the cold reservoir in the large water tank 26.

In a method of operating the thermal therapy system 10 with closed-loop biometric control, the method may include one or more or any of the steps of: donning the garment 60 and positioning the biometric sensor 114 on the user; activating a therapy mode (cold, hot, or contrast); sensing biometric parameters of the user using the biometric sensor 114; and automatically adjusting the water temperature, session duration, or other operating parameters responsive to the sensed biometric parameters. For example, the method may comprise reducing the water temperature or terminating the session when the user's heart rate exceeds a threshold value, or extending the session duration when the user's heart rate variability indicates an inadequate physiological response.

### Exemplary Parameters

The following exemplary parameters are provided to illustrate preferred operating ranges for the thermal therapy system 10, though the invention is not limited to these specific values.

The water tank system may have a total water capacity of approximately 10 to 15 liters, with the large water tank 26 comprising approximately two-thirds of the total capacity and the small water tank 28 comprising approximately one-third of the total capacity. In one example, the total water capacity is approximately 12 liters, with the large water tank 26 holding approximately 8 liters and the small water tank 28 holding approximately 4 liters.

The water cooling mode may cool the water to a setpoint temperature of approximately 1° C. to 5° C., with a preferred setpoint of approximately 2° C. At this setpoint temperature, water circulated through the garment 60 produces a skin temperature experience similar to immersion in a cold water bath at approximately 8° C. to 12° C.

The water heating mode may heat the water to a setpoint temperature of approximately 35° C. to 45° C., with a preferred setpoint of approximately 40° C. At this setpoint temperature, water circulated through the garment 60 produces a comfortable warming sensation without risk of burns.

A typical cold therapy session may last approximately 3 to 15 minutes, with a preferred session duration of approximately 5 to 10 minutes. A typical hot therapy session may last approximately 10 to 30 minutes.

The air bags 72 may be inflated to a pressure of approximately 5 to 50 millibars gauge, with a preferred pressure of approximately 10 to 30 millibars gauge. This pressure range provides adequate force to press the water bags 74 into contact with the user's body without causing discomfort.

The water flow rate through the garment 60 may be approximately 1 to 10 liters per minute, with a preferred flow rate of approximately 3 to 6 liters per minute. This flow rate provides rapid transit of temperature-conditioned water through the water channels 76 while maintaining acceptable pressure losses.

While specific embodiments of the present invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention, which is to be given the full breadth of the appended claims and any and all equivalents thereof.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶ 16, other aspects may likewise be embodied as a means-plus-function claim, or in other forms, such as being embodied in a computer-readable medium. (Any claims intended to be treated under 35 U.S.C. § 112, ¶ 16 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A thermal therapy system comprising:
a temperature control unit comprising:
a refrigerant loop;
a water tank system;
at least one water pump configured to circulate water between the water tank system and a wearable garment; and
an air pump; and
a wearable garment configured to be worn by a user, the garment comprising:
at least one garment portions that includes an air bag, and a water bag disposed inwardly of the air bag;
wherein the water bag defines a plurality of water channels configured to receive temperature-conditioned water from the temperature control unit;
wherein the air bag is configured to receive pressurized air from the air pump and to inflate to press the water bag into contact with the user's body; and
a plurality of hoses fluidly connecting the temperature control unit to the garment, including at least one water inlet hose, at least one water outlet hose, and at least one air hose.

2. The thermal therapy system of claim 1, wherein the temperature control unit further comprises an ice-making coil disposed within the water tank system, the ice-making coil being in thermal communication with the refrigerant loop such that ice forms around the ice-making coil when refrigerant is directed therethrough.

3. The thermal therapy system of claim 2, wherein the temperature control unit is configured to route return water from the garment through a region containing ice formed around the ice-making coil.

4. The thermal therapy system of claim 1, wherein the water channels are configured as water highways extending in a substantially vertical direction when the garment is worn by the user, with one or more water inlets positioned at a first location on the garment and one or more water outlets positioned at a second location on the garment that is spaced from the first location.

5. The thermal therapy system of claim 4, wherein the one or more water inlets are positioned at a first location proximate the user's shoulders and the one or more water outlets are positioned proximate the user's feet.

6. The thermal therapy system of claim 1, wherein the air bag and the water bag are joined together at intermittent weld points disposed at spaced intervals, such that inflation of the air bag presses the water bag into contact with the user's body without substantially occluding the water channels.

7. The thermal therapy system of claim 1, wherein the refrigerant loop comprises a compressor, a four-way reversing valve, a heat sink, and a plate heat exchanger, and wherein the four-way reversing valve is configured to reverse the direction of refrigerant flow to switch the temperature control unit between a cooling mode and a heating mode.

8. The thermal therapy system of claim 1, wherein the at least one water pump comprises an internal circulation pump configured to circulate water between the water tank system and a plate heat exchanger, and at least one garment pump configured to circulate water through the garment.

9. The thermal therapy system of claim 8, wherein the at least one garment pump comprises a first garment pump configured to circulate water through a left body portion and a left arm sleeve of the garment, and a second garment pump configured to circulate water through a right body portion and a right arm sleeve of the garment.

10. A method of operating a thermal therapy system, the method comprising the steps of:
(a) cooling water in a water tank system of a temperature control unit;
(b) after the water is cooled, a user donning a wearable garment fluidly connected to the temperature control unit, the garment comprising a water bag defining a plurality of water channels and an air bag disposed outwardly of the water bag;
(c) inflating the air bag to press the water bag into contact with the user's body;
(d) after the air bag is at least partially inflated, circulating the cooled water from the water tank system through the water channels of the water bag;
(e) after completion of a therapy session, removing water from the water bag; and
(f) deflating the air bag.

11. The method of claim 10, further comprising, prior to step (b), forming ice around an ice-making coil disposed within the water tank system, and during step (d), return water from the garment is routed through a region containing the ice formed around the ice-making coil such that heat is extracted from the return water by the ice.

12. The method of claim 10, wherein step (e) comprises purging residual water from the water bag by directing pressurized air into the water channels to force the residual water back to the water tank system.

13. The method of claim 12, wherein the pressurized air in step (e) enters the water channels through one or more inlet connections and forces the residual water toward one or more outlet connections.

14. The method of claim 10, wherein during step (d), cooled water enters the garment at one or more inlet connections located at a first portion of the garment and flows through the water channels toward outlet connections located at a second portion of the garment.

15. A wearable garment for a thermal therapy system, the garment comprising:
at least one garment portion comprising:
an air bag; and
a water bag disposed inwardly of the air bag;
wherein the water bag defines a plurality of water channels;
shoulder inlet or outlet connections positioned proximate the user's shoulders for receiving or returning temperature-conditioned water;
foot inlet or outlet connections positioned proximate the user's feet for receiving or returning water; and
wherein the air bag and the water bag are joined together at intermittent weld points disposed at spaced intervals, such that inflation of the air bag presses the water bag into contact with the user's body without substantially occluding the water channels.

16. The garment of claim 15 wherein the water channels are configured as water highways extending in a substantially vertical direction when the garment is worn by the user.

17. The garment of claim 15, comprising a plurality of garment portions, including a right body portion, a left body portion, a right arm portion, and a left arm portion, and wherein the right arm portion and the left arm portion are attached to the right body portion and the left body portion, respectively, at shoulder connections.

18. The garment of claim 17, wherein the left body portion and the right body portion each define at least four water channels extending from a shoulder inlet region to a foot outlet region.

19. The garment of claim 15, wherein the air bag includes a plurality of internal weld lines that extend generally horizontally across the air bag when the garment is worn.

20. The garment of claim 18, wherein the left arm portion is fluidly connected to at least one of the water channels on the left body portion, and wherein the right arm portion is fluidly connected to at least one of the water channels on the right body portion.
